(19) United States
(12) Patent Application Publication
(10) Pub. No.: US 2025/0318955 A1
(43) Pub. Date: Oct. 16, 2025

(54) SYSTEM AND METHODS FOR TREATING GLAUCOMA WITH LASER PULSES

(71) Applicant: EyeX Solutions Inc., San Juan Capistrano, CA (US)

(72) Inventor: Christopher Horvath, San Juan Capistrano, CA (US)

(73) Assignee: EyeX Solutions Inc, San Juan Capistrano, CA (US)

(21) Appl. No.: 19/054,841

(22) Filed: Feb. 15, 2025

Related U.S. Application Data
(60) Provisional application No. 63/553,967, filed on Feb. 15, 2024.

(51) Int. Cl. A61F 9/008; A61F 9/009

(52) U.S. Cl. CPC A61F 9/00814; A61F 9/009; A61F 2009/00851; A61F 2009/00861; A61F 2009/00891

(57) ABSTRACT

Laser-based ophthalmic systems and methods can be used to treat glaucoma and other conditions of the eye. The laser system can be used to form openings or partial-thickness channels in the trabecular meshwork to promote aqueous humor outflow. Scanning approaches provided herein can create either fully perforating “full hole” ablations or partial-disruption “soft holes.”

### CROSS-REFERENCE TO RELATED APPLICATIONS

This application claims priority under 35 U.S.C. 119(e) to, and the benefit of, U.S. Provisional Application No. 63/553,967, filed Feb. 15, 2024, the entirety of which is incorporated herein by reference.

### BACKGROUND

Lasers have been used for several decades in the treatment of glaucoma. The two most common laser treatments for primary open angle glaucoma (“POAG”) are argon laser trabeculoplasty (“ALT”) and selective laser trabeculoplasty (“SLT”). See, for example, U.S. Pat. Nos. 3,884,236, 8,066,696, 5,549,596, and 6,319,274. They work by applying laser pulses into the trabecular meshwork (located in the anterior angle of the eye).

These laser pulses are focused to about 50 micrometer diameter for ALT and about 400 micrometer for SLT. Those laser spots are targeted to lay over the trabecular meshwork and increase outflow through the treated meshwork area into the Schlemm's canal by modulating the tissue properties. In both procedures, at least 90 degrees of the anterior angle of the eye is treated with typically 180 degrees and 50 to 100 laser pulses (each pulse is applied to a new target zone-treatment area).

The working mechanism for ALT is blanching of the trabecular meshwork that increases the outflow by stretching the trabecular meshwork between the blanched (laser treated areas). The ALT laser, with a typical setting of 600 mW and 0.1 s pulse duration (at 514 nm or 532 nm), causes a thermal tissue interaction.

In SLT treatment, the laser causes cavitation bubbles in the target tissue due to its shorter pulse duration of about 3 nanoseconds and higher peak power (created by pulse energies of about 0.3 mJ to 1.6 mJ).

Both procedures have a good success rate in that they increase aqueous humor outflow to create a substantial drop in intraocular pressure of about 20%. Both procedures can be performed in minutes with a simple slit lamp procedure in the office (no operating room required). In both procedures, the eye does not need to be opened (i.e., they are non-invasive procedures, and no incisions are needed). Therefore, the treatment risks and complication rates are minimal.

### SUMMARY

In accordance with at least some embodiments disclosed herein is the realization that there are significant problems associated with these procedures. For example, these procedures do not work effectively in all patients. Also, even in the successful cases, the effect wears off over the course of a few years (1-3 years) and the intraocular pressure (“IOP”) of the eye rises over time. The procedure can only be repeated once with ALT and two or three times with SLT, because after being repeated, the tissue damage created in the trabecular meshwork ultimately reduces or altogether prevents any further IOP lowering effect.

A less frequently used laser procedure called excimer laser trabeculostomy (“ELT”) uses an excimer laser pulse (with a wavelength in the UV range) to drill holes into the trabecular meshwork. See, for example, U.S. Pat. App. Nos. 2008/0082078 and 2004/0082939. Because complete openings are created to Schlemm's canal (unlike ALT and SLT), the IOP lowering effect is similar or better than ALT or SLT. Also, only a few open holes need to be drilled with ELT versus 50-100 treatment zones in a typical ALT or SLT procedure. Some studies further suggest that the ELT effect is longer lasting then ALT or SLT due to some observed long-term patency of those holes. Furthermore, ELT might be repeated more often since a smaller area of the trabecular meshwork is treated each time.

In accordance with at least some embodiments disclosed herein is the realization that the downside of ELT is that UV wavelength light does not penetrate the cornea and aqueous humor, and therefore, the laser can only be applied to the trabecular meshwork in a sterile operating room, where the eye is opened and a fiber probe is inserted into the anterior chamber, all of the way up to the trabecular meshwork.

In recent years, the effectiveness of having one or multiple holes in the trabecular meshwork (connecting to Schlemm's canal) has also been demonstrated with several implants, placed through the trabecular meshwork that creates a connection of the anterior chamber to Schlemm's canal, bypassing the meshwork. Another surgical method to remove, cut or incise part or all of the trabecular meshwork is called goniotomy or trabeculotomy often done by inserting a mechanical device into the eye. See, for example, U.S. Pat. App. Nos. 2012/0071809 and 2007/0276316. However, in accordance with at least some embodiments disclosed herein is the realization that such procedures are invasive procedures that require a sterile operating room and use an implant or a surgical tool inside the eye.

Another approach to drain aqueous humor out of the anterior chamber has been successfully demonstrated by implanting a drainage tube through the scleral spur region and into the suprachoroidal space. See, for example, U.S. Pat. App. No. 2011/0098629. This is, however, also an invasive procedure that requires a sterile operating room and uses an implant.

Most recently, there have been animal tissue studies and initial human trials done by ViaLase applying ultrashort photodisruptive laser pulses to the trabecular meshwork with good success. See, for example, Vialase in Opthtalmology Times, Sep. 21, 2021.

However, in accordance with at least some embodiments disclosed herein is the realization that large challenges and areas of improvement remain within the area of delivering ultrashort laser pulses to the trabecular meshwork, due to the existing complexity and high cost of such laser systems and also due to the difficulty in visualizing and targeting the anterior angle tissues of the eye.

According to some embodiments, the present inventions described herein relate to new devices and methods that overcome the limitations and challenges of the above-noted conventional procedures and therefore allow the creation of holes, open sections and channels (goniotomy, trabeculomy or trabeculostomy) in the trabecular meshwork and other places in the anterior angle area of an eye also referred to as irido-corneal angle of an eye or within this patent specifications referred to as the target area in the angle. When this procedure is performed as a standalone procedure, it can be done in a non-invasive approach and therefore avoid any opening of the eye. The herein described systems and methods can reduce complexity, improve visualization and targeting of the angle tissue and can be repeated many times over as needed.

Referring briefly to FIG. 28, the anterior angle area of a normal eye is shown in a detailed side cross-sectional view. The trabecular meshwork 7907 can be separated in a lower pigmented section 7910 and the upper non-pigmented section 7905. The lower pigmented part is thicker than the upper part and measures about 150 um to 300 um in thickness. The trabecular meshwork can comprise three separately defined layers. Starting deeper inside the eye and moving outwards, there is the uveal meshwork 7930, the corneoscleral meshwork 7935 and the juxtacanalicular tissue layer 7940. A healthy trabecular meshwork lets the aqueous humor flow through from the anterior chamber 7925 into Schlemm's canal 7945. As the liquid propagates through these three tissue layers it encounters growing flow resistance until it finally flows through the inner wall of Schlemm's canal 7945 and then through Schlemm's canal until it exits through a collector channel 7925. Schlemm's canal goes around the entire 360 degrees of the eye angle but is not a complete open tube and contains many septums and elastic bands, which create a pumping contraction to push the aqueous humor along. There are also narrowing segments along is circumference. Flow of aqueous humor will therefore not be equal in both directions once the aqueous humor reaches Schlemm's canal. Furthermore, the on-average about 25-30 collector channels around the eye circumference are not equally placed in all eye quadrants but have a higher density in the nasal region of the eye. Therefore, some embodiments of the herein described trabecular meshwork opening creation via laser can advantageously performed in the nasal region of the eye.

In accordance with at least some embodiments disclosed herein is the realization that the trabecular meshwork cannot be considered a simple shaped open tube. Especially in advanced stages of glaucoma Schlemm's canal starts to collapse as shown in FIG. 27, at element 7945, and this makes targeting Schlemm's canal more difficult. Some embodiments disclosed herein can comprise a 3D imaging system in the laser treatment system that helps identifying the shape, size and location of Schlemm's canal and the other eye features. In all cases, Schlemm's canal is mostly located behind the pigmented part of the trabecular meshwork 7910, which represents the lower part of the angle tissue area, and therefore, some embodiments of the laser system described herein can advantageously focus on visualizing, identifying and targeting that region for best access to Schlemm's canal.

Referring to FIGS. 28 and 29, at least some embodiments disclosed herein relate to the realization that the target area volume 7950 can be defined by the following dimensions: (1) Z-axis: from most inner, first trabecular meshwork layer (uveal) 7930 through all layers and into Schlemm's canal 7945; (2) Y-axis: from the scleral spur 7915 or relative to that up to Schwabe's line 7900 or to the upper end region of the pigmented part of trabecular meshwork 7910 where the tissue transitions from pigmented 7910 to non-pigmented 7905 part of the trabecular meshwork or relative to either; and (3) X-axis: along the circumference of the anterior angle as wide of an arc opening in the eye as desired, including multiple holes.

As further described herein, the present disclosure addresses these and other problems by providing systems, devices, and methods that can incorporate one or more of the following features.

For example, a laser system is provided herein that can form openings or partial-thickness channels in the trabecular meshwork to promote aqueous humor outflow when photo disruptive laser pulses are used. Some embodiments include a pulsed laser source (femtosecond to nanosecond duration), beam-expanding and shaping optics, one or more scanning units (e.g., rotating wedges, tilted parallel plates, or offset lenses) for generating circular, ring, or partial-coverage spot patterns, and a patient interface with an integrated gonioscopic lens or camera-goniolens assembly. These scanning approaches can create either fully perforating “full hole” ablations or partial-disruption “soft holes.” The latter preserve significant tissue volume while achieving tissue remodeling or flow enhancement.

Several embodiments feature low-density scanning patterns, Z-axis translation for volumetric ablation, multi-speed spinners, and advanced diagnostic imaging modules (e.g., OCT, two-photon detection or confocal (microscope) signal feedback) for real-time positioning, thickness measurement, or tissue differentiation.

In some embodiments, the system can comprise digital cameras and specialized mirrors, or entire camera-goniolens subassemblies, in order to allows direct or angled visualization of the anterior angle.

In some embodiments, one or multiple integrated digital cameras can be used in the delivery system path for fs laser, therefore, eliminating the need for a slit lamp or surgical microscope or any other integrated visual microscope. Furthermore, the camera can be mounted next to goniolens and, in some embodiments, integrate with and within the goniolens as a detachable subunit of the delivery system or as a complete separate device.

Optionally, in some embodiments, when a camera is integrated with to goniolens, then that subsystem can be detached and used independently for diagnostic purposes.

In some embodiments, a standalone goniolens can comprise an attached and/or integrated camera and one or more various advanced lighting systems.

In some embodiments, a camera-integrated goniolens can be provided that provides visualization for pre-operative care, immediately before laser fires (final confirmation of targeting treatment location), during laser firing and post op visualization of the angle tissue.

In some embodiments, the goniolens can comprise a mirrored goniolens. In some embodiments, a direct goniolens can be integrated with digital cameras.

In some embodiments, the goniolens can comprise one or more illumination systems. For example, the goniolens can comprise one or more illumination systems that utilize infrared, visible wavelengths, low light, customizable/specific wavelength settings, and/or direct light to the retina for iris contraction or dilation.

In some embodiments, the goniolens or systems disclosed herein can be configured as a multiple delivery system with integrated 3D visualization systems.

In some embodiments, systems and methods provided herein can utilize an optical coherence tomography (“OCT”) imaging beam system over the target area and/or through the limbus area to locate and visualize Schlemm's canal. This OCT system may be integrated with the laser treatment system and/or can be used as a standalone diagnostic system that is used without surgery. This OCT system can be used pre-op, immediate before firing, during treatment, and/or post-op.

In accordance with some embodiments, the system and methods disclosed herein can be configured to provide automatic detection and/or selection of a targeting area. Further, in some embodiments, the systems and methods disclosed herein can be configured to provide a user interface having the functionality to enable a user to manually select a laser target area via a crosshair on a computer screen, which can show a camera visualization of the target area.

Some of the advantages of the presently disclosed systems and methods include improved scanning flexibility, fewer aberrations, insensitivity to misalignment, smaller mechanical footprints, and real-time tissue imaging. The ability to target partial-thickness zones or the entire trabecular meshwork fosters a personalized, repeatable therapy. Additionally, a variety of pulse energies (tens to hundreds of microjoules) and scanning densities enables delicate “soft hole” remodeling or robust “full hole” creation. Collectively, these innovations expand laser-based glaucoma treatment options and reduce the need for invasive surgical procedures or mechanical implants.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is an overview diagram of a laser system, showing a laser source, one or more beam-expanding/scanning units, an optional imaging module, and a patient interface unit, in accordance with some embodiments.

FIG. 2 is a schematic view of a laser system that illustrates a beam-shaping and combining unit in relation to an imaging and patient interface unit, in accordance with some embodiments.

FIG. 3 is a schematic view of a laser system that comprises an optional subunit that can comprise one or more imaging, diagnostic, and/or guidance unit(s), an aiming beam, and/or 3D imaging modalities (e.g., OCT, second harmonic, etc.), in accordance with some embodiments.

FIG. 4 is a schematic view of a laser system that comprises a combined camera-goniolens configuration with a focusing/scanning unit, in accordance with some embodiments.

FIG. 5 is a schematic view of a laser system that comprises a patient interface unit (goniolens) and beam combining unit in a colinear imaging/camera unit, in accordance with some embodiments.

FIG. 6 is a side cross-sectional view of an eye illustrating a target region for laser treatment, in accordance with some embodiments.

FIG. 7 is a side cross-sectional view of an eye illustrating an anterior chamber angle with indicated treatment zones, in accordance with some embodiments.

FIG. 8 is a side cross-sectional view of an eye illustrating a patient interface lens resting on a cornea for angle access, in accordance with some embodiments.

FIG. 9 is a side cross-sectional view of an eye illustrating an alternative lens design for contacting the cornea, in accordance with some embodiments.

FIG. 10 is a side cross-sectional view of an eye illustrating an interface assembly with an extra glass piece for use as a sterile barrier, in accordance with some embodiments.

FIG. 11 is a side cross-sectional view of an eye illustrating a goniolens with a peripheral flange and suction ring, in accordance with some embodiments.

FIG. 12 is a side cross-sectional view of an eye illustrating a goniolens with a partial cutout over the limbus region, in accordance with some embodiments.

FIG. 13 is a side cross-sectional view of an eye illustrating a visualization system that utilizes a mirror-based angle visualization system with two outgoing beams, in accordance with some embodiments.

FIG. 14 is a side cross-sectional view of an eye illustrating a visualization system that utilizes a variation of a mirror-based angle visualization system using a mirrored goniolens approach, in accordance with some embodiments.

FIG. 15 is a side cross-sectional view of an eye illustrating a visualization system that utilizes a single-mirror visualization system and approach that contacts a cornea, in accordance with some embodiments.

FIG. 16 is a side cross-sectional view of an eye illustrating a visualization system that utilizes a rotational scanning approach with a vertical axis and side-based OCT beam, in accordance with some embodiments.

FIG. 17 is a side cross-sectional view of an eye illustrating a visualization system that utilizes a camera module integrated behind a partially transmissive mirror, in accordance with some embodiments.

FIG. 18a is a side cross-sectional view of an eye illustrating a visualization system that utilizes a camera with a mirrored goniolens, in accordance with some embodiments.

FIG. 18b is a schematic top view of a focusing/scanning unit comprising an offset rotation feature to perform a scanning procedure, in accordance with some embodiments.

FIG. 19 is a side cross-sectional view of an eye illustrating a visualization system that utilizes a handheld mirrored goniolens camera arrangement, in accordance with some embodiments.

FIG. 20 is a side cross-sectional view of an eye illustrating a visualization system that utilizes a camera-lens assembly positioned over the cornea, in accordance with some embodiments.

FIG. 21 is a side cross-sectional view of an eye illustrating a visualization system that utilizes a handheld camera-lens block with a limbus cutout, in accordance with some embodiments.

FIG. 22 is a side cross-sectional view of an eye illustrating a visualization system that utilizes a combined camera-goniolens unit with a rotating handle and at least one imaging subunit, in accordance with some embodiments.

FIG. 23 is a side cross-sectional view of an eye illustrating a visualization system comprising one or more integrated camera-lens submodules in an exploded view, in accordance with some embodiments.

FIG. 24 is a side cross-sectional view of an eye illustrating a visualization system that utilizes a dual approach with a goniolens, a rotating mirror, and an OCT imaging beam entering the eye, in accordance with some embodiments.

FIG. 25 is a side cross-sectional view of an eye illustrating a visualization system that utilizes a rotatable mirror unit above a goniolens for 360° angle scanning, in accordance with some embodiments.

FIG. 26 is a side cross-sectional view of an eye illustrating a visualization system that utilizes another rotational mirror arrangement, in accordance with some embodiments.

FIG. 27 is a side cross-sectional view of a trabecular meshwork of an eye having a collapsed Schlemm's canal and meshwork layers, in accordance with some embodiments.

FIG. 28 is a side cross-sectional view of a trabecular meshwork of an eye illustrating another angle cross-section for scanning over a collapsed or partially open Schlemm's canal, in accordance with some embodiments.

FIG. 29 is a partial top view of an anterior angle of an eye with a target zone, in accordance with some embodiments showing several laser scan pattern.

FIG. 30a is a plan view of a circular scanning pattern with arcs over a grid representing the trabecular meshwork, in accordance with some embodiments.

FIG. 30b is a plan view of a scanning pattern with partial arcs in a larger circle, in accordance with some embodiments.

FIG. 30c is a plan view of a full circular scanning pattern with continuous scanning as the beam translates laterally creating a capsule shaped pattern, in accordance with some embodiments.

FIG. 30d is a plan view of a high density capsule shaped pattern on the right used for hole cutting and a low density capsule shaped pattern on the left used for “soft hole” procedure, in accordance with some embodiments.

FIG. 31 is a side cross-sectional view of a trabecular meshwork of an eye illustrating target meshwork layers showing partial thickness cuts, in accordance with some embodiments.

FIG. 32 is a schematic view of a beam-intensity profile and scanning lens offset method to perform a scanning procedure, in accordance with some embodiments.

FIG. 33 is a schematic view of an optical system capable of forming a circular and/or capsule shaped scanning pattern, in accordance with some embodiments.

FIG. 34 is a schematic view of another optical system capable of forming a circular and/or capsule shaped scanning pattern, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

According to some embodiments disclosed herein, systems, devices, and methods are provided whereby a laser can be used to create openings or channels to open sections through the trabecular meshwork of an eye to allow aqueous humor to flow therethrough, thereby serving to provide a reduction in IOP. In some embodiments, the openings or channels can provide fluid communication between the anterior chamber and at least part of Schlemm's canal and/or collector channels of the trabecular meshwork, thereby permitting a reduction in the IOP of the eye. In this manner, the present disclosure provides effective tools and procedures that can revolutionize the treatment of glaucoma, overcoming the various disadvantages and drawbacks of the prior art systems and methods discussed above.

### Laser Systems

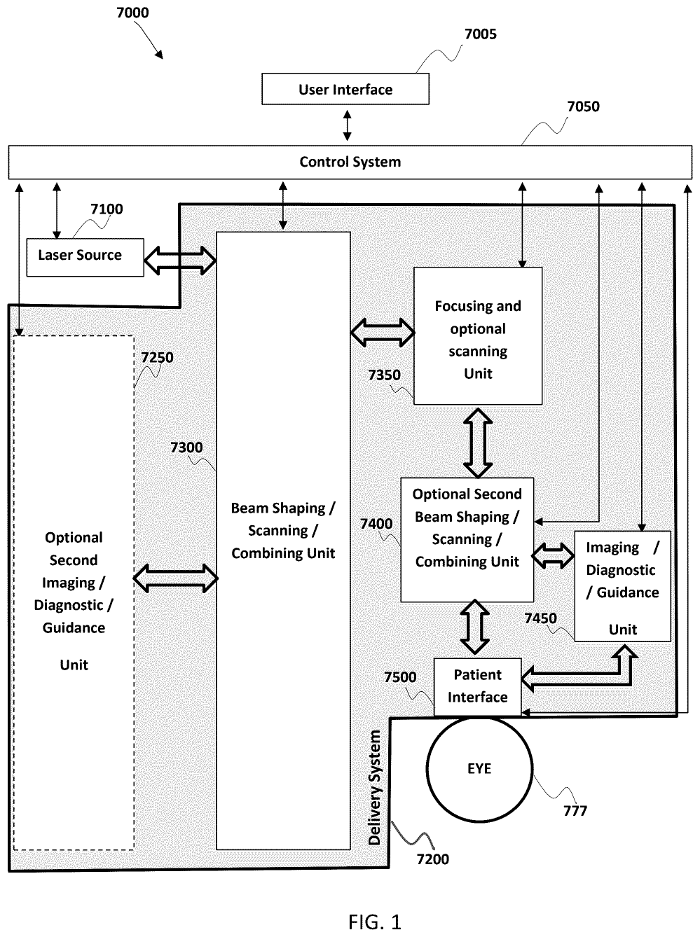

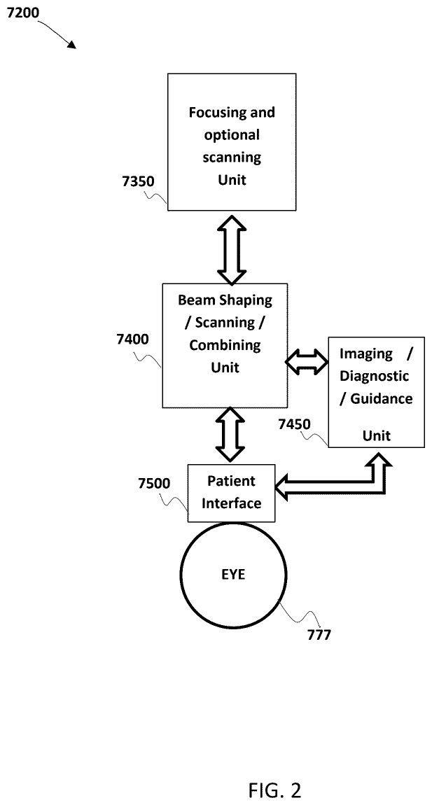

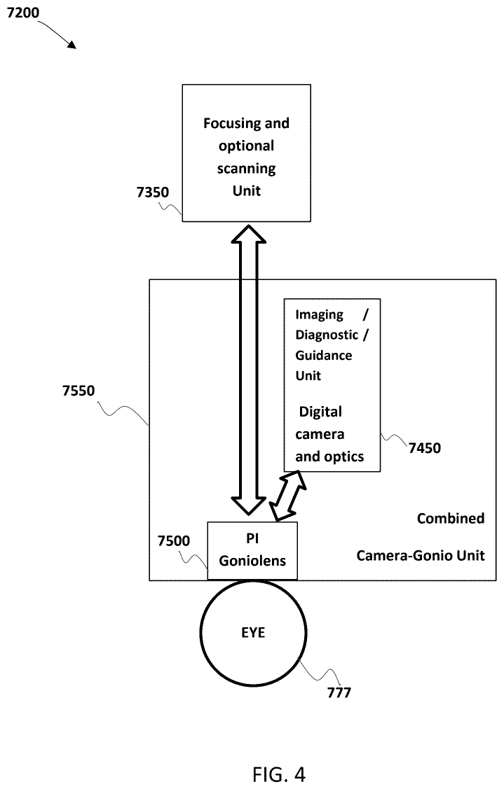

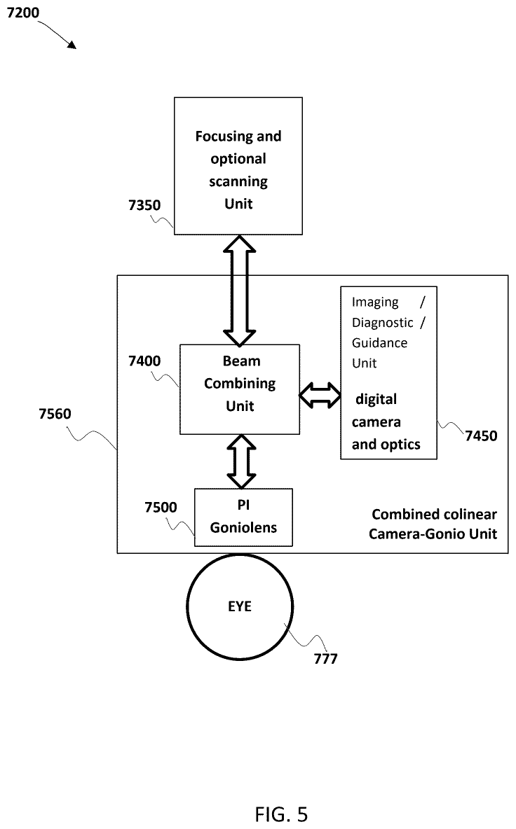

Referring now to the figures, FIGS. 1-5 illustrate various overview diagrams of embodiments of a laser system, showing a laser source, one or more beam-expanding/scanning units, an optional imaging module, and a patient interface unit, in accordance with some embodiments. The laser system 7000 of FIG. 1 can comprise a user interface 7005, a control system 7050, a laser source 7100, and a delivery system 7200. For sake of brevity, each of these subsystems or sub-units of the laser system 7000 can comprise one or multiple units. For example, the laser source 7100 can include either one laser source or multiple laser sources. The same is true with the user interface 7005, which can include either a single user interface or multiple, separate user interfaces.

The user interface 7005 can comprise at least one input (such as a foot or hand switch) and at least one feedback device. The input can comprise an input foot or hand switch (operated by a human foot or hand) with a single switch or a multi-control input foot or hand switch with several switches and adjustment input abilities such as multidimensional joystick capabilities or other input systems. The foot or hand switch may optionally comprise one or more feedback (output) mechanisms.

The feedback mechanism can serve to inform the operator of various states and parameters of the laser system before, during and/or after the treatment procedure. In accordance with some embodiments, the feedback mechanism can comprise various components configured to provide information and feedback to the user. For example, the feedback mechanism can provide visual, tactile, and/or audio feedback to the user.

The feedback mechanism can provide at least one visual feedback signal, such as lights with various colors, brightness and blinking patterns. For example, red, green and yellow indication lights static on, off or blinking.

Further, the feedback mechanism can utilize at least one tactile feedback system that provides tactile feedback, such as mechanical vibrations of various strength, frequency and timing. For example, if the laser system is in a warning or error state, the foot or hand switch could start vibrating to inform the operator.

Moreover, the feedback mechanism can utilize at least one audio feedback system that provides audible feedback, such as beeping or other tone generators or voice feedback with a specific message for the operator. For example, the system may say “System ready” or “Treatment complete,” based on the status of the system.

In accordance with some embodiments, the user interface 7005 can also comprise at least one display device. The display device can be integrated into the foot or hand switch. The display device can also comprise one or several computer screens or touch screens for input and output of visualization, other data and commands.

In accordance with some embodiments, the user interface 7005 can also comprise a computer, a keyboard, mouse or any other computer user interface.

Referring still to FIG. 1, the control system 7050 can comprise at least one computer and/or electronics boards (e.g., a PCB) to power, control, and process input and output data of the laser sources, the user interface and all subsystems of the delivery system 7200.

The laser source 7100 can comprise a (first) laser engine that produces short laser pulses with a pulse duration between 100 femtosecond and 10 nanoseconds, a pulse energy between 1 uJ (MicroJoule) and 500 uJ, a pulse repetition rate between 10 Hz and 100 kHz and a wavelength between 350 nm and 1600 nm.

In some embodiments, the laser source 7100 can also comprise a second laser engine with the same parameters as above. Optionally, the second laser engine can be controlled independently of the first laser engine.

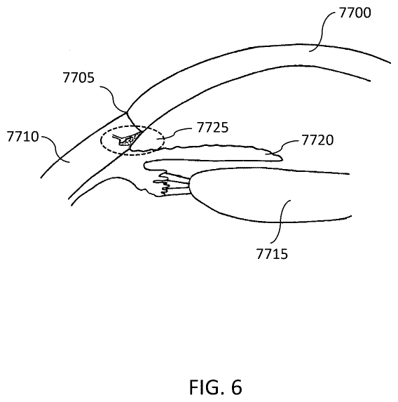

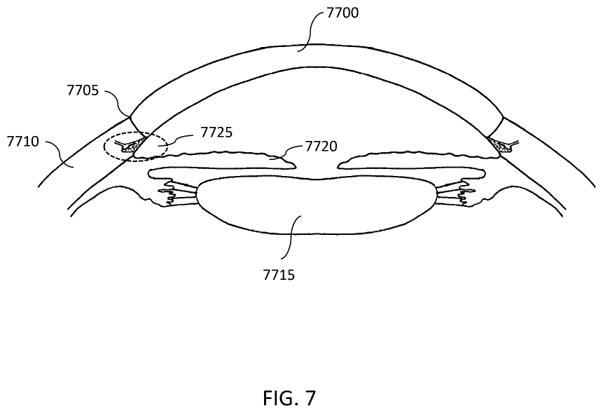

Using features and capabilities of the various embodiments of the systems and methods disclosed herein, a target region 7725 of the eye can be treated, as illustrated in FIGS. 6 and 7. Further details of the laser system 7000 are provided below.

### Components of the Delivery System

As shown in FIGS. 1-5, in accordance with some embodiments disclosed herein, the delivery system 7200 can comprise one or more imaging units (that may be configured to facilitate imaging, diagnostics, and/or guidance), one or more beam shaping units (that may be configured to facilitate beam shaping, scanning, and/or being combining), and/or one or more focusing units (that may be configured to facilitate beam focusing and/or scanning). In the present disclosure, these units may be referred to individually by their potential functions or generally, for example, as “imaging unit,” “beam shaping unit,” or “focusing unit,” and the recitation (or lack thereof) of potential functions need not limit the embodiments to require (or exclude) the unit from performing that functionality. Further, the embodiments illustrated in FIGS. 1-5 provide examples of configurations in which the delivery system incorporates one or two of each of the above-noted units, and it may be desirable to vary the configurations in order to achieve desired results, as discussed herein with respect to other aspects of the laser system.

Referring to the embodiments shown in FIGS. 1-5, the delivery system 7200 can comprise an imaging unit 7450, a beam shaping unit 7300, and a focusing unit 7350, an optional beam shaping unit 7400, an optional imaging unit 7250, and a patient interface unit 7500, in accordance with some embodiments.

### Imaging Unit Features

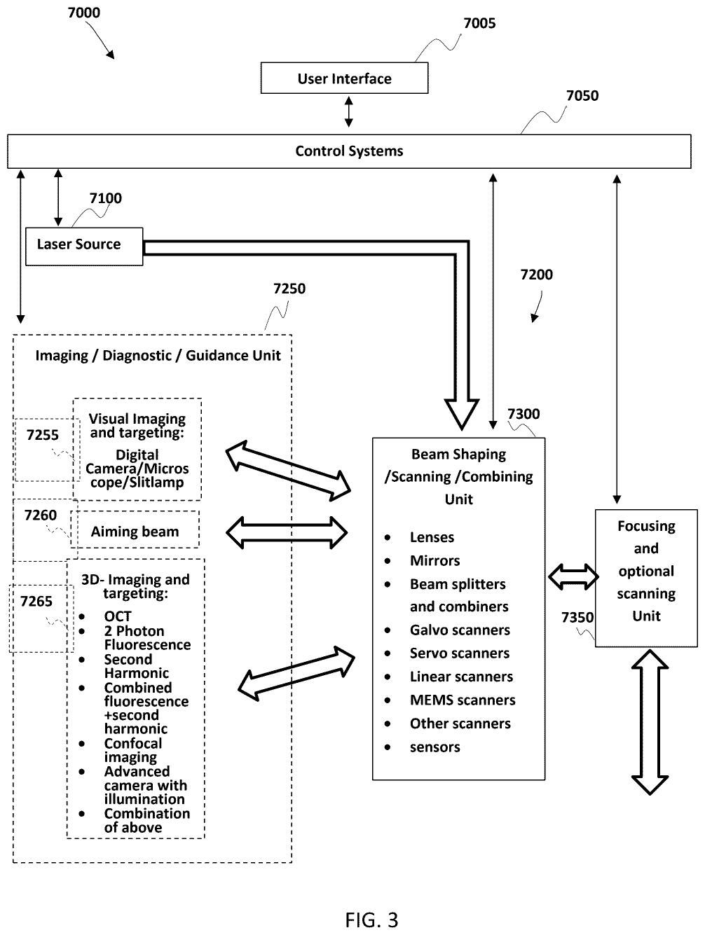

In some embodiments, the delivery system 7200 of the laser system 7000 can comprise an imaging unit (shown as imaging/diagnostic/guidance unit) 7250, as shown in FIG. 3. The optional imaging unit 7250 can comprise one or more imaging, diagnostic, and/or guidance unit(s), an aiming beam, and/or 3D imaging modalities (e.g., OCT, second harmonic, etc.).

The imaging/diagnostic/guidance unit 7250 can comprise a visual imaging unit 7255, an aiming beam light source 7260, and a 3D imaging and targeting device 7265. The 3D imaging and targeting device can comprise one or more of an OCT imaging system, a two-photon detection system, a second harmonic beam detection system, a confocal imaging system, a digital camera, and/or an illumination system.

In accordance with some embodiments, the visual imaging unit 7255 can comprise one or multiple digital cameras for visible and/or non-visible (e.g., infrared) light to capture and process target area visualization data, an integrated visual microscope for direct view of the target area by the operator, a surgical microscope that is integrated with the delivery system, a slit lamp system that is integrated with the delivery system or any combination thereof. The visual imaging unit 7255 can advantageously be used for visualization and targeting the laser.

In some embodiments, the aiming beam light source 7260 can provide a single red or other wavelength laser beam that has a strong focusing angle (e.g., less than 10 degrees) on the target tissue, thereby providing a means to aim for the target tissue laterally (left-right and up-down), as well as an ability to set and calibrate the z-distance which is longitudinal to the laser path and along the axis connecting the proximal extend to the distal extend of the targeted tissue area.

For example, the z-calibration can be done by placing the focus (smallest spot) of the aiming laser onto or near a defined surface in the eye, such as the proximal surface of the trabecular meshwork. Another version can comprise two or more converging laser beams that meet into one point which is a defined and calibrated point in the z-axis of the laser delivery system. Moving the delivery system components such that the meeting point of these aiming beams falls on the target tissue (e.g. the proximal surface of the trabecular meshwork) will provide z-calibration of the laser system.

In some embodiments, the OCT imaging system, which can optionally be used in the 3D imaging and targeting device 7265, can use an optical coherence tomography beam for achieving a 3D visualization of the target area.

In some embodiments, the two-photon detection system, which can optionally be used in the 3D imaging and targeting device 7265, can be used to analyze fluorescence feedback light that gets emitted by the target area due to the two-photon interaction of the high peak power treatment laser pulse with the target tissue. This fluorescence feedback diagnostic system is based on the principles of two-photon microscopy.

In two-photon microscopy, the fluorescence photons that travel back from the target area through the eye, the goniolens and into a designated detector in the system have a photon energy that is less than two times the treatment laser photon energy. In other words, the wavelength of the fluorescence photons being detected is shorter than the wavelength of treatment laser but longer than one-half of the wavelength of the treatment laser. The feedback signal, which is typically very small, travels through a narrow optical bandwidth filter designed to transmit the fluorescence wavelength and mostly block other wavelengths. The feedback signal then lands on a dedicated photodetector. The fluorescence signal photons are created by an interaction of the treatment laser focus and the eye-tissue located at that focus. A small part of these signal photons makes it back through the optics system and onto the dedicated photodetector. The signal strength from this photodetector is then recorded and saved together with the momentary target location coordinates of the laser focus. As the laser scans through the target area, the computer and software can then create a 3D imaging map with these individual data points. In accordance with some embodiments, this fluorescence feedback beam can be used in the systems, devices, and methods disclosed herein to create various imaging and calibration data such as laser energy, in particular laser threshold for photodisruptive breakdown, laser pulse duration, laser focusing parameters for example focus size and focus position, optical aberration performance, target tissue surface detections, target tissue depth penetration and target tissue thickness data, target tissue type identification and/or other tissue interactions with the treatment laser. One of the surprising benefits and advantages of some embodiments disclosed herein is that this imaging scan of the target area by the treatment laser can be done with reduced laser power settings and just prior to the actual laser treatment.

In some embodiments, the second harmonic beam detection system, which can optionally be used in the 3D imaging and targeting device 7265, can utilize some or all of the features as described above in the two-photon microscopy based detection system, but instead of being based on fluorescence feedback from the two-photon interaction, the second harmonic beam detection system can be based on a non-linear optical interaction of two photons of the treatment laser beam with the target tissue. Thus, the second harmonic beam detection system can create a frequency-doubled photon feedback signal that has half the wavelength of the treatment laser. Every aspect of the above two-photon fluorescence feedback applies to this method as well and will not be repeated here for brevity. Notably, the primary difference is a small shift in the feedback wavelength and the signal strength.

The combined two-photon diagnostic system (two-photon fluorescence and second harmonic), which can optionally be used in the 3D imaging and targeting device 7265, as described above, wherein both the fluorescence feedback photons and the second harmony photons are both detected either together in one detector or in two separate detectors using different optical bandwidth filters. The different signal strength variations from focal point to focal point adds additional image data and resolution to analyze the tissue layers and improved targeting.

Referring still to FIG. 3, the confocal imaging system, which can optionally be used in the 3D imaging and targeting device 7265, can be based on the principles of confocal microscopy. This system allows a high-resolution depth penetrating (3D) visualization of the target tissue in the eye.

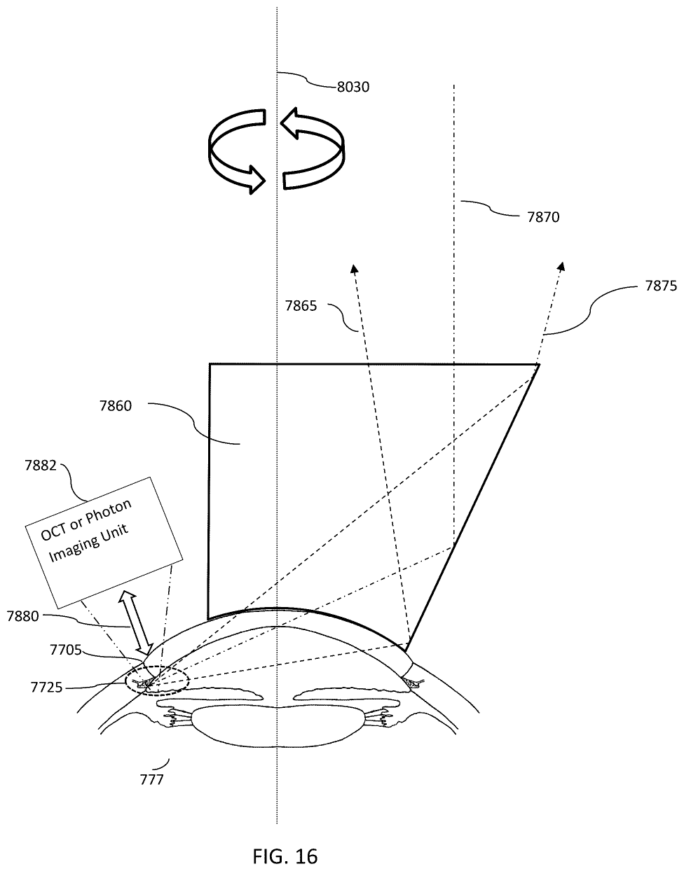

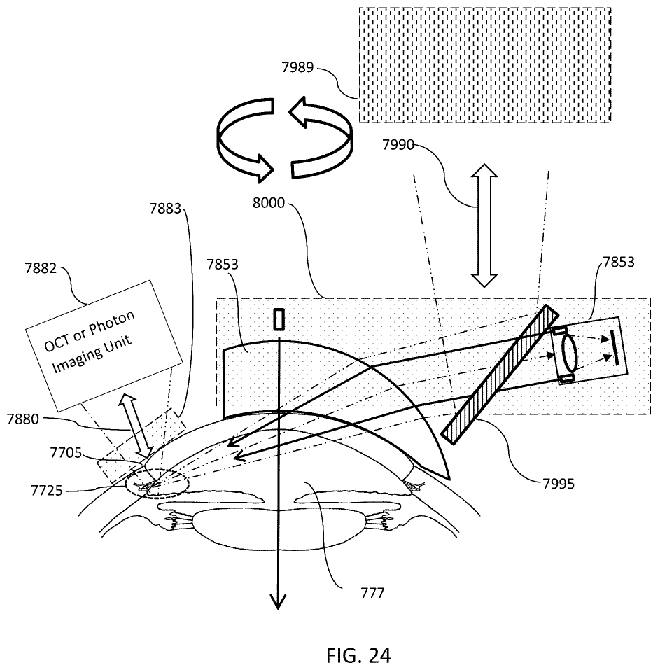

The 3D imaging and targeting device 7265 can also optionally include an imaging system that uses a digital camera. The imaging system can use a digital camera can comprise one or multiple features, as described below in the section Patient Interface Unit: Digital Camera Features. See the embodiments of FIGS. 24-26 as one example (which generally represent modified versions of the system shown in FIG. 16, further incorporating a camera and illumination unit in combination with a mirror component).

The 3D imaging and targeting device 7265 can also optionally include an illumination and/or camera system with one or multiple features as described below in the section Patient Interface Unit: Digital Camera Features.

### Beam Shaping Unit Features

In some embodiments, the delivery system 7200 of the laser system 7000 can comprise beam shaping optics, such as a beam shaping unit 7300 for shaping, scanning, and/or combining one or more beams. For example, the beam shaping optics can comprise a beam shaping and/or scanning and/or combining unit. The beam shaping unit 7300 can comprise one or any combination of beam shaping optics, scanning optics, and/or optical beam combining elements, for example, to combine the laser treatment beam and an alignment laser beam to propagate colinear through the focusing unit and into the eye.

The beam shaping optics can be configured to modify the laser beam parameters of the treatment laser as it propagates through the delivery system towards the focusing unit. This can include control of the laser beam diameter, convergence angles, astigmatism control and other laser beam parameter. This unit also contains additional optics elements for some or all of the above-described second imaging/diagnostic/guidance unit 7250 and its imaging, diagnostics and guidance beams that are propagating through the delivery system 7200.

The beam scanning unit can comprise scanning optics that can be configured to create various treatment laser scanning patterns. This can include optical galvo, servo scanners, gimbal mount scanners, micro-scanners, MEMS and other electromechanically driven optical scanners. The scanning optics unit can also contain all the optical scanners that are additionally required for the above-described second imaging/diagnostic/guidance unit 7250 and its imaging, diagnostics and guidance beams.

In accordance with some embodiments, the scanning optics can be configured to perform a scanning procedure. As noted above, the scanning procedure can create various treatment laser scanning patterns. Such patterns can comprise full or partial ring scanning, circle scanning, arc scanning, and overlapping circular patterns, examples of which are discussed herein. Moreover, exemplary scanning patterns, optical systems, rotating optics, and related methods are also discussed and illustrated in copending U.S. Pat. App. No. 63/750,772, filed on Jan. 29, 2025, the entirety of which is incorporated herein by reference.

The beam shaping unit 7300 can also comprise all required optical beam combining elements, for example, to combine the laser treatment beam and an alignment laser beam to propagate colinear through the focusing unit and into the eye. The beam shaping unit 7300 can also comprise various laser and diagnostic beam sensors to monitor the laser system parameters.

### Focusing Unit Features

A focusing and optional scanning unit 7350 is shown in FIGS. 1-5, and can comprise a focusing optics assembly to focus the treatment laser beam and all the other imaging, diagnostic and guidance beams towards the target tissue in the eye. The focusing and optional scanning unit 7350 can comprise one or multiple optics elements, such as lenses. In some embodiments, the focusing and optional scanning unit 7350 can advantageously comprise a single aspherical lens.

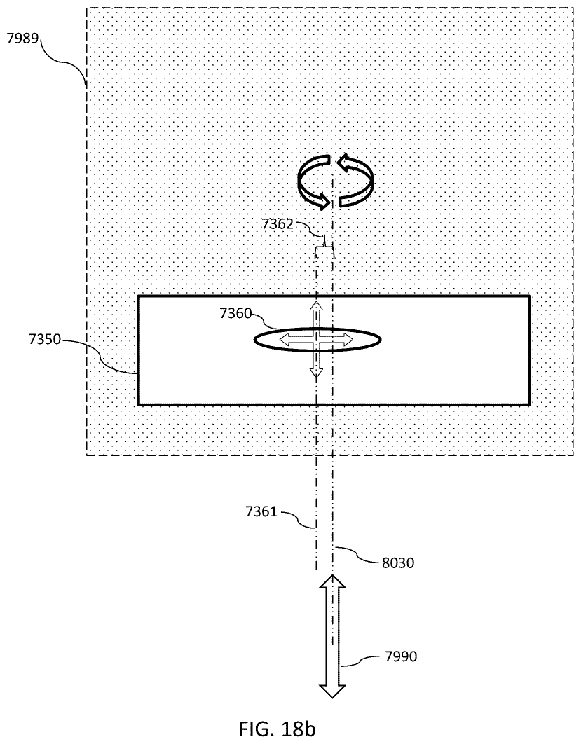

In the embodiment shown in FIG. 18b, the unit 7350 contains a scanning system wherein the treatment laser beam 7990 is scanned by spinning and/or translating one or more focusing optics or components of a focusing optics assembly 7360. The focusing optic can be in the form of a lens, parallel plate, or wedge through which the laser beam 7990 can pass and be shifted thereby. The focusing optic can define a central symmetry axis 7361 that is parallel to but offset from the central optical axis 8030 of the delivery system by the amount illustrated as element 7362.

In some embodiments where the focusing optic(s) is spinning, the focusing optic(s) of the focusing optics assembly 7360 can define a central spinning axis that is identical with the central optical axis 8030 of the delivery system. The focusing optic(s) can be oriented such that the incident angle of the central laser treatment beam 7990 is orthogonal (90 degrees) to the spinning plane of the focusing optic(s).

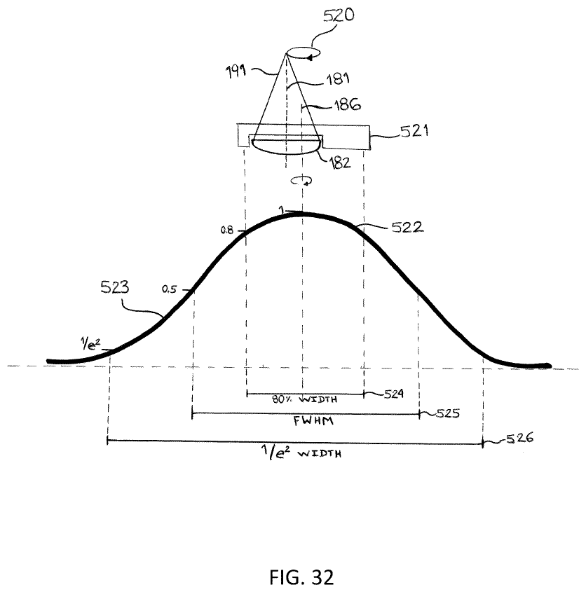

In accordance with some embodiments, all these scanning modes further optionally including a z-axis translation drive/scan to also scan in the longitudinal z-axis which is parallel to the central optical axis 8030. For example, FIG. 32 shows another illustration of a spinning offset lens 182 (focusing optic) circling around a central optical axis 186, having an offset between the axis 181 and 186, an overfilled gaussian input laser beam 523 such that the input laser beam coming here from the bottom up into the spinning lens is stationary and the resulting laser focus through the spinning lens creates a circular scan 520.

Optionally, as shown generally in FIGS. 24-26, a scannable/tiltable mirror can be mounted posterior to the focusing optics and therefore closer to the target tissue. That mirror can be driven by an electromechanical device and being used as a primary or secondary scanner to perform the desired laser scan pattern in the target tissue. That mirror can advantageously be mounted in a gimbal mount or comprise a micromirror/MEMS-based mirror unit or gimbal and MEMS-based scanning mirror.

### Beam Shaping Unit Optional Features

An optional second beam shaping/scanning/combining unit 7400 can comprise one or more scanning units, optical interfaces, and/or beam combining units.

The scanning unit can comprise an additional or primary scanning unit of any type as described in the units 7300 and 7350. The scanning unit having the unique feature of being placed posterior/after the focusing optics unit.

The one or more optical interfaces can comprise at least one element, including the above-described scanning unit can be configured for shaping the laser and other beams further.

The beam combining unit can overlap and/or combine the laser treatment beam with the various optics beams coming in from the imaging/diagnostic/guidance unit 7450 that is placed nearby, for delivering all the overlapping and/or combined beams into the eye.

### Imaging Unit Optional Features

An optional imaging/diagnostic/guidance unit 7450 can comprise one or more of any or all the subsystem units from the optional second imaging unit 7250 discussed above, a slit lamp unit, an aiming beam unit, and/or a digital camera unit.

In some embodiments, the imaging unit 7450 can advantageously comprise a slit lamp unit that can generate a visualization beam and an optional aiming beam unit that can generate an aiming beam. In such embodiments, the slit lamp visualization beam can have a path that can enter directly into the patient interface unit and even if the slit lamp visualization beam is not exactly colinear with the treatment laser beam and the optional aiming beam, the slit lamp visualization beam can enter the beam combining unit 7400 and become colinear with the treatment laser beam (and the optional aiming beam).

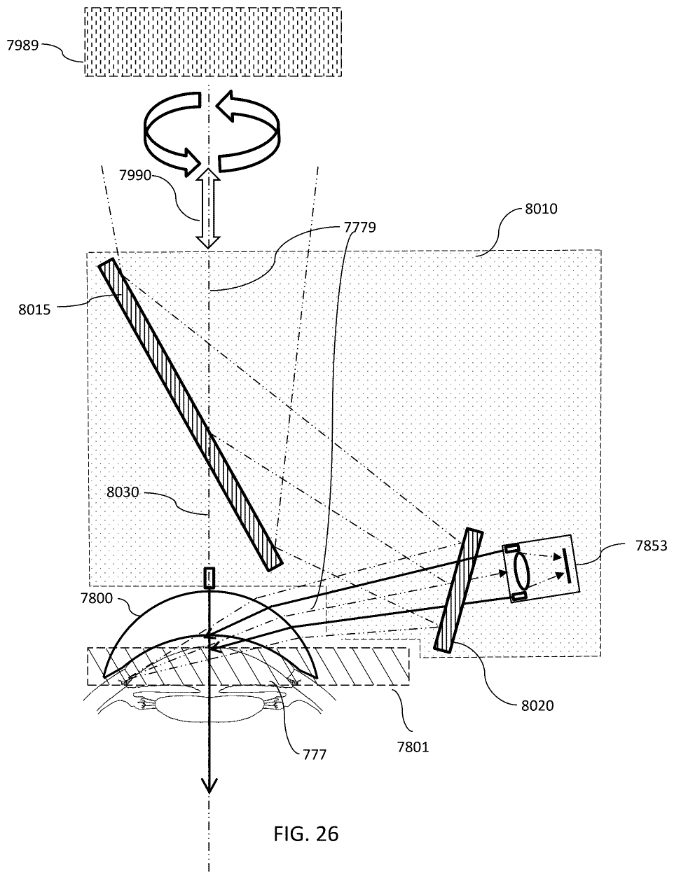

Further, in some embodiments, the imaging unit 7450 can also advantageously comprise at least one digital camera with integrated imaging optics and its visualization beam being routed via a beam combining element of beam combining unit 7400 between the eye and the camera. Alternatively, the camera can be mounted in a slightly offset way such that the target tissue imaging beam is captured by the camera in a small angle relative to the treatment laser and therefore does not require a beam combining element and does not travel through unit 7400. The camera and its optics and the entire imaging unit 7450 can be connected to the patient interface unit 7500 and, if present, also be connected to the optional second beam shaping/scanning/combining unit 7400; and such embodiments, the imaging unit 7450 and the patient interface unit 7500 can become one integrated unit, or the imaging unit 7450, the beam combining unit 7400, and the patient interface unit 7500 can become one integrated unit. An example embodiment is illustrated, for example, in FIG. 26 where the patient interface unit or goniolens unit 7801 is combined with a mirror unit 8010.

Furthermore, the imaging unit 7450 can also advantageously be configured such that the at least one digital camera can have one or multiple features of the camera described below in the section Patient Interface Unit: Digital Camera Features.

### Patient Interface Unit: Goniolens Features

The patient interface unit can comprise a goniolens. For example, the patient interface unit 7500 can comprise a goniolens having a concave bottom surface to closely fit a human cornea to create an optical interface without minimizing or eliminating any air gap between the goniolens and the cornea. A liquid or gel is optionally applied between the goniolens and the cornea for better contact.

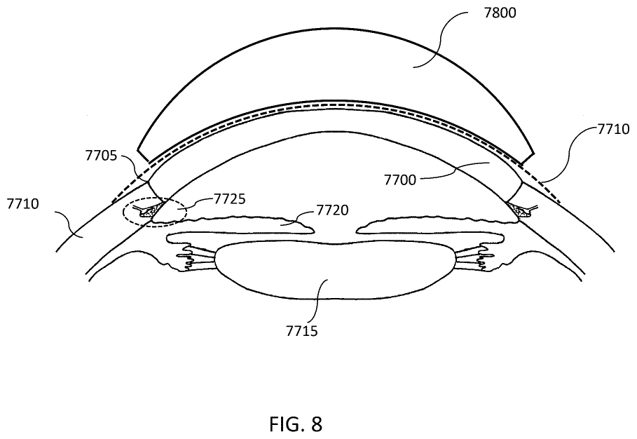

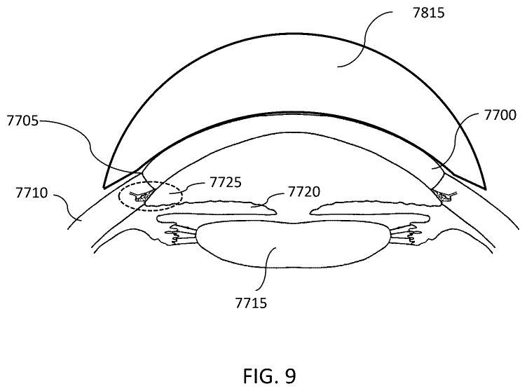

Optionally, the goniolens can also or alternatively comprise a spherical or aspherical convex curvature on the top side to create further focusing and aberration control of the laser treatment beam and diagnostic and visualization beams. Example designs are seen in FIG. 8, 7800 and FIG. 9, 7815.

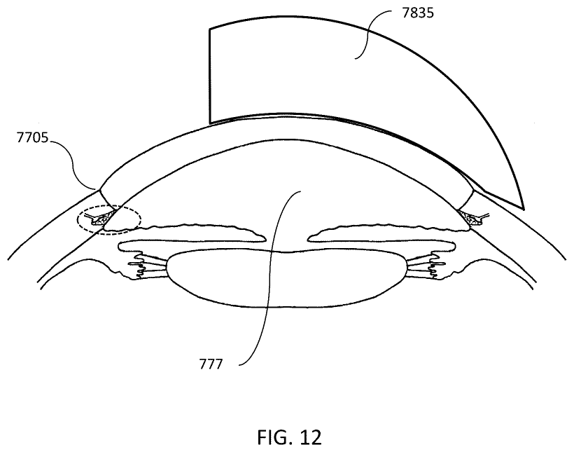

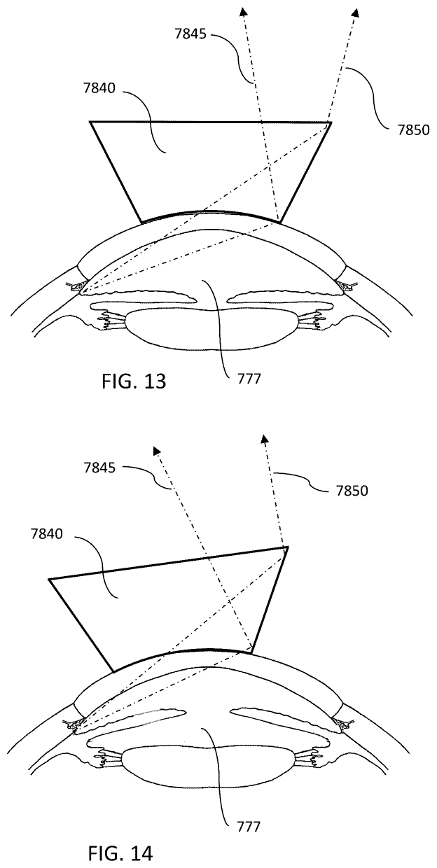

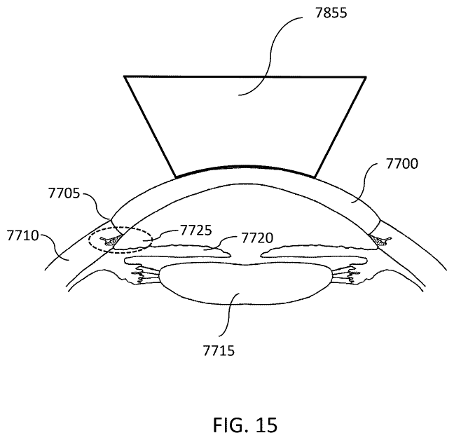

Some aspects of the goniolens are shown in FIG. 12-15, which illustrate various features and uses of a goniolens, including partial cutout or peripheral shapes that permit the use of OCT or other imaging beams, and mirror-based angle visualization systems with two outgoing beams (see elements 7845, 7850), in accordance with some embodiments, as in FIGS. 13 and 14. FIG. 15 shows a visualization system that utilizes a single-mirror visualization system and approach 7855 that contacts a cornea, in accordance with some embodiments.

In some embodiments, optionally, the goniolens can also or alternatively comprise at least one side that has a flat or curved mirror surface and the top surface of the goniolens substrate is either flat or curved. See, for example, FIG. 15 (see element 7855) and FIG. 16 (see element 7860). The mirror angle to the horizontal plane can be between 60 deg and 70 deg.

Further, optionally, the goniolens can also or alternatively comprise a substrate that has a cutout over the limbus area 7705 where the angle target tissue area 7725 lays, as generally illustrated in FIG. 12 (see element 7835) and FIG. 16 (see element 7860). This cutout may not affect the laser beam entering the goniolens and eye since the laser entry into the goniolens is sufficiently shifted away from this part of the goniolens that there is not interference, as generally illustrated in FIGS. 16 and 24. In two embodiments FIG. 16 and FIG. 24, this uncovered segment of the cornea and the limbus right over the treatment area 7725 in the eye can be used for additional diagnostic, imaging and treatment beams entering the eye from that side 7880, including entering through the transition zone around the limbus 7705 that can comprise the lowest part of the cornea 7770 and the beginning part of the scleral tissue 7710 to probe, visualize and treat the anterior angle tissue region 7725 specifically Schlemm's canal, the trabecular meshwork layers, the iris, the iris root, the ciliary body tissue, the scleral spur region and the suprachoroidal tissue layers in the eye.

Furthermore, optionally, the goniolens can also or alternatively comprise a cutout or body shape having partial coverage of the cornea such that a gap or cutout in the body is large enough to allow access above the target region for an OCT diagnostic and visualization beam 7880, a two-photon microscopy beam or another imaging beam that can penetrate the partial scleral tissue layers. For example, the cutout or gap can be formed by creating an asymmetric body shape of the goniolens. The cutout or gap is illustrated, for example, in the embodiments shown in FIGS. 16, 24, and 28, where an OCT diagnostic and visualization beam 7880 can be used and pass to the eye without obstruction of the goniolens.

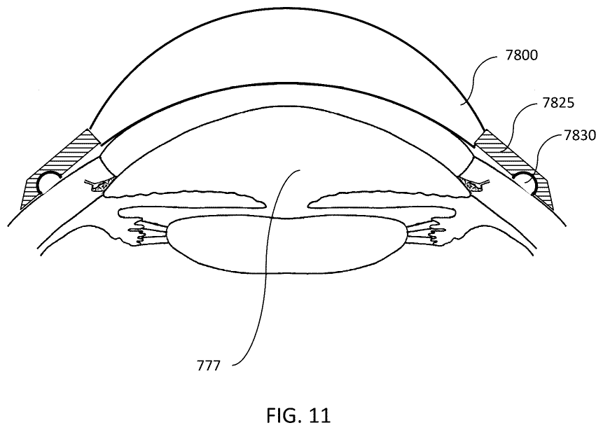

In accordance with some embodiments, the goniolens 7800 can optionally comprise a flange on the outer rim, as shown but not limited to FIG. 11, 7825, that is shaped to fit tight over the transition zone between the cornea and sclera and thereby covers the limbus and continues over the sclera. In some embodiments, the flange 7825 can have one or more sections cut out to allow access to the limbus in some parts of the eye. This allows for example access to some parts of the eye limbus area where a diagnostic beam is placed, as illustrated in FIG. 24.

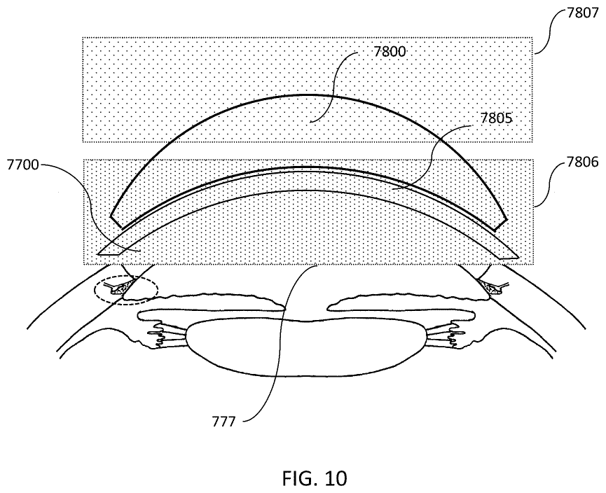

In some embodiments, the patient interface can incorporate any of the patient interface features noted herein and further comprise a sterile clear substrate or optic component of a soft material 7710, as shown in FIG. 8, or a hard glass material 7805, as shown in FIG. 10. The optic component can be placed between the cornea 7700 and the goniolens 7800 such that it provides a sterile barrier between the eye 777 and the patient interface 7500.

In some embodiments, the patient interface unit can incorporate any of the patient interface features noted herein and further be configured such that the substrate or optic component 7710 comprises a thin, clear flexible sheet (e.g., less than about 300 um thick) that allows all laser and other beams to travel through without creating significant aberrations. The optic component 7710 can be placed on the eye 777 before the goniolens 7800 is place on the eye. Optionally, a clear liquid or gel can be applied below and/or above the optic component 7710 to make better contact to the eye and the goniolens. The optic component 7710 in one configuration can be sized such that it only covers the cornea larger that it overlaps over the limbus or beyond covering at least part of the scleral/conjunctival tissue or being large enough to cover the entire eye area of the patient. Further, the optic component 7710 can optionally be connected and integrated with a sterile drape that is being place over at least part of the patient's head.

In some embodiments, the patient interface unit can incorporate any of the patient interface features noted herein and further be configured such that an additional optics piece 7805 is made from glass such as fused silica, Fluoropolymers, BK7 or any other glass that provides a good enough quality to not significantly degrade the laser and other beams transmission. The glass can advantageously be used in a sterile condition and optionally be for single use. The glass can be shaped convex on the bottom such that it will make sufficient good contact with the cornea 7700 such that no significant laser and other desired beam degradation occurs. The glass can have an upper surface being convex or flat such that it allows docking to the lower side of the goniolens 7800 without any significant or no airgaps therebetween. Optionally, a clear gel or liquid can be applied to the eye to improve contact with the cornea and/or the goniolens.

In some embodiments, the patient interface unit can incorporate any of the patient interface features noted herein and be configured such that a glass 7700 is mounted to a structure 7806, as shown in FIG. 10. The structure 7806 can be connected and fixed to a goniolens mounting structure 7807 above and thereby being placed on the eye 777 together with the entire patient interface unit or where it is placed on the eye first and then at a later stage in the procedure being docked to the mounting structure 7807 of the goniolens mount such that it connects in a final aligned fixed position via a docking procedure. Optionally, the glass 7805 and its mounting structure 7806 can have a built-in flange 7825, as shown in FIG. 11, and/or an integrated suction ring 7830 and/or a peripheral flange 7825 that provides further stabilization of the structure.

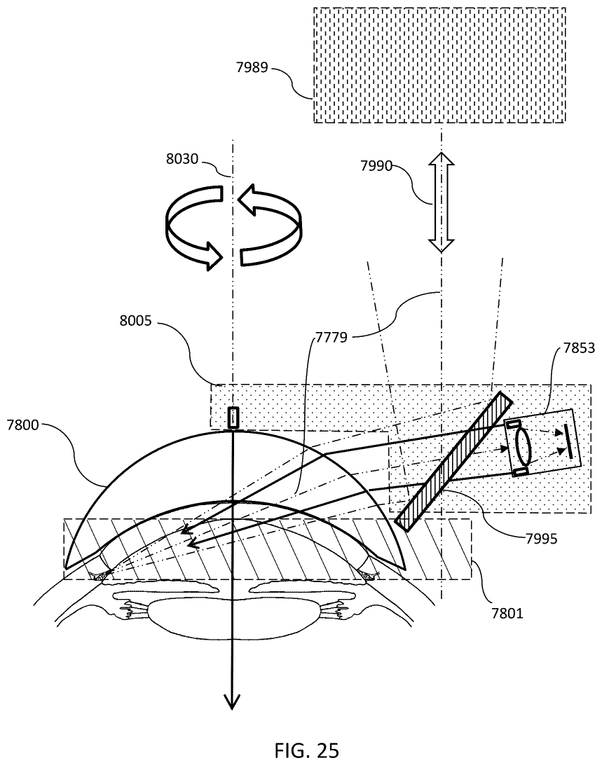

In some embodiments, the patient interface unit can incorporate any of the patient interface features noted herein and further comprise a mounting structure 8000, as shown in FIGS. 24 and 25, that extends around or is coupled to the goniolens 7853. The mounting structure 8000 can allow the goniolens 7853 to be removably connected to the rest of the delivery system 7200 of the laser system 7000 to permit the goniolens 7853 to be attached or detached by the operator.

In some embodiments, the suction ring 7830 can be integrated with the flange 7825 or a standalone suction ring to allow a suction stabilized connection between the patent interface 7500 and the eye.

Further, in some embodiments, the goniolens unit can be rotated by 360 degrees before or after being placed on the eye, therefore allowing access to the entire anterior angle of the eye, as generally illustrated in FIGS. 16-26.

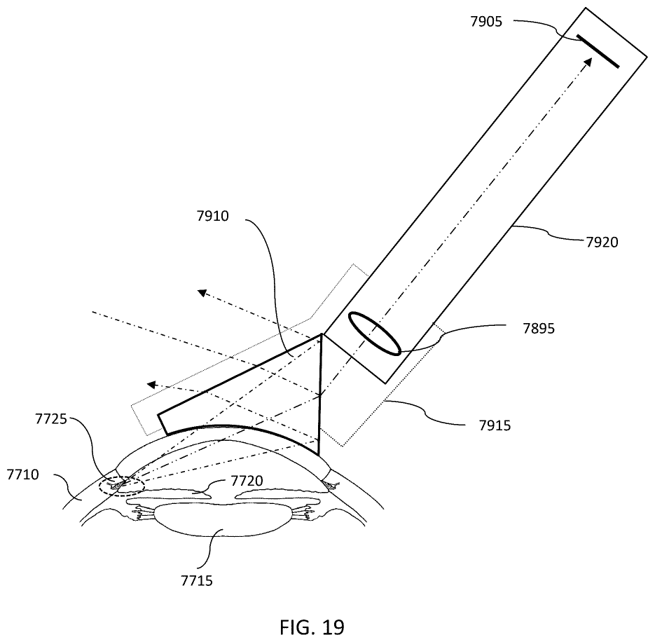

For example, in some embodiments, the goniolens unit is not docked or connected to the rest of the delivery system and can be rotated independently either by handheld means or by using a separate mounting with its own motorized actuators, as generally shown in FIGS. 19, 20 and 21.

In some embodiments, the patient interface can incorporate any of the patient interface features noted herein and further be configured such that the goniolens has a 360-degree, fully symmetric optic. Some embodiments of the goniolens unit can be docked to the rest of the delivery system during the treatment. Furthermore, the beam path can be able to rotate 360 degrees by rotating the last routing mirror unit 8005 (see FIG. 25), containing one routing mirror 7995 or rotating the last routing mirror unit 8010 (see FIG. 26), containing two routing mirrors 8015 and 8020, before the goniolens in a circular path around a central eye axis 8030 such that the treatment beam rotates 360 degrees and enters the goniolens 7800 in a symmetric way. The last routing mirror 8005 or mirror unit 8010 can be rotated while the goniolens unit 7801 and the mirror(s) unit is docked to the rest of the delivery system including a rotational joint. Furthermore, this configuration can include an optional second rotational joint between the goniolens unit 7801 and the mirror(s) unit 8005 or 8010 to allow the mirror(s) unit being rotated around the goniolens unit 7801, while the goniolens unit is being fixed to the eye via suction and while all units are mechanically docked or permanently connected to the rest of the delivery system.

In some embodiments, as generally illustrated in FIG. 25, the patient interface unit can incorporate any of the patient interface features noted herein and further comprise a mirror unit 8005 and/or a camera and illumination unit 7853 (described further below in the section Patient Interface Unit: Digital Camera Features). The camera and illumination unit 7853 and/or the mirror unit 8005 can be coupled and/or permanently connected to a goniolens unit 7801 to form a combined unit (referred to herein as combined unit 8005+7801). The combined unit 8005+7801 can comprise any of the features or components described herein for the mirror unit 8005, the camera and illumination unit 7853, or the goniolens unit 7801. In the illustrated embodiment of FIG. 25, the combined unit 8005+7801 can be detached from the rest of the delivery system 7989 prior to the treatment procedure.

Further, in some embodiments, the combined unit 8005+7801 can be placed and moved on the eye, advantageously by hand, establishing a connection between the goniolens 7800 and the eye 777.

In accordance with some embodiments, this movement of the combined unit 8005+7801 along the eye can facilitate performance of the treatment procedure by permitting the user to align the central imaging beam path 7779 with the desired target area in the anterior angle of the eye using the optional camera unit 7853 or other visual means. Following this alignment, the combined unit 8005+7801 can optionally be fixed to the eye using a suction device similar or identical to the configuration in FIG. 11 or continues to be handheld in place while the rest of the delivery system 7989 now being placed over this combined unit 8005+7801 and aligned such that the central laser treatment beam 7990 becomes colinear or close to colinear with the central imaging beam path 7779. The rest of the delivery system 7989 can then be docked and connected to the combined unit 8005+7801 or remain unconnected in this pre-aligned position. Thereafter, the targeting alignment of the laser system can be finalized using imaging and diagnostic data to auto-align the laser target or perform alignments via operator input. After completing the final targeting alignment, the laser treatment can be initiated and completed.

FIG. 26 illustrates an embodiment in which a mirror unit 8015, including an optional camera and illumination unit 7853 (described further below in the section Patient Interface Unit: Digital Camera Features), can be permanently connected to a goniolens unit 7801 and where the combined unit 8015+7801 is detached from the rest of the delivery system 7989 prior to the treatment procedure. The combined unit 8005+7801 can be placed and moved on the eye, advantageously by hand, establishing a connection between the goniolens 7800 and the eye 777 and aligning through translation and rotation of the combined unit 8005+7801 the central imaging beam path 7779 to the desired target area in the anterior angle of the eye using the optional camera unit 7853 or other visual means. Following this alignment, the combined unit 8005+7801 can optionally be fixed to the eye using a suction device similar or identical to the configuration in FIG. 11 or continues to be handheld in place while the rest of the delivery system 7989 now being placed over this combined unit 8005+7801 and aligned such that the central laser treatment beam 7990 becomes colinear or close to colinear with the central imaging beam path 7779. The rest of the delivery system 7989 can then be docked and connected to the combined unit 8005+7801 or remain unconnected in this pre-aligned position. Thereafter, the targeting alignment of the laser system can be finalized using imaging and diagnostic data to auto-align the laser target or perform alignments via operator input. After completing the final targeting alignment, the laser treatment can be initiated and completed.

### Patient Interface Unit: Digital Camera Features

In some embodiments, the patient interface unit can incorporate any of the patient interface features noted herein and further comprise a digital camera and imaging optics. These digital camera features can be incorporated into the patient interface (e.g., goniolens) in a variety of configurations.

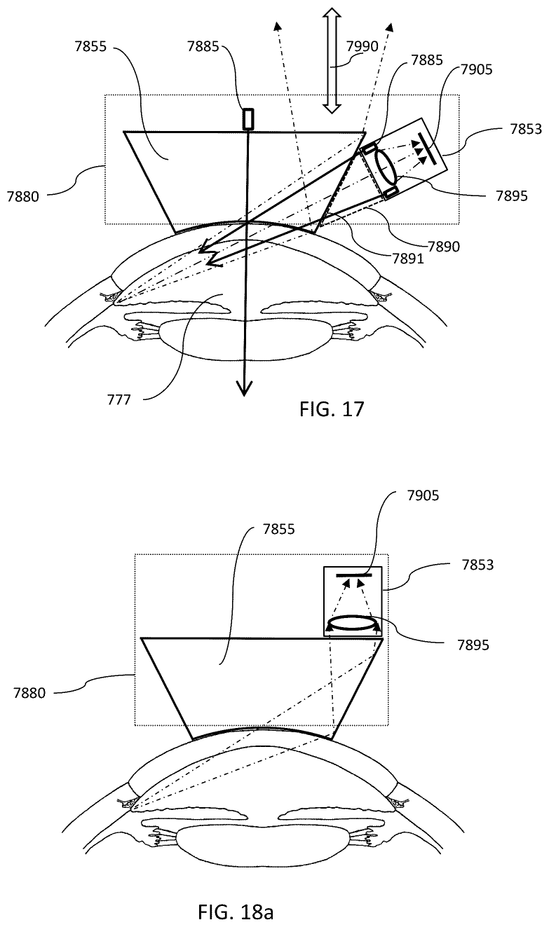

For example, FIGS. 17, 18a, 24, 25, and 26 show a goniolens 7855, 7853 or 7800 laying on an eye, the treatment and diagnostic beams 7990 are coupled into the goniolens and eye via a mirror. The mirror can be at least partially transmissible to the wavelength range that the digital camera is sensitive to, a digital camera with imaging optics can be mounted behind that mirror such that the target region of the laser system is imaged through the goniolens, the mirror, the camera imaging optics 7895 and onto the digital camera sensor 7905. The camera unit 7853 housing can be connected or integrated to a goniolens unit mounting. See, for example, FIG. 17, 7880. The connected or integrated camera unit can be either fixed or can be adjustable in three dimensions to allow for mechanical alignments such that a calibrated vision of the target area relative to the treatment and other beams can be reflected by the mirror is achieved. Therefore, allowing lateral x, y alignment and calibration as well as focus, defined as z here, adjustments.

In some embodiments, the patient interface unit can incorporate any of the patient interface features noted herein and further be configured such that the patient interface unit is fixed connected to the rest of the delivery system mounting. For example, FIG. 24, the patient interface unit 8000 can be fixed connected to the rest of the delivery system 7989.

In some embodiments, the patient interface unit can incorporate any of the patient interface features noted herein and further be configured such that the patient interface unit is initially decoupled from the rest of the delivery system mounting, and then after it has been placed on the eye with or without suction, can be docked to the rest of the delivery system and then creating a fixed combined unit 8005+7801. For example, FIG. 26, the patient interface subunits 7801 and 8010 initially can be decoupled from each other and from the rest of the delivery system 7989 and then after alignment, all be docked and connected to each other 7801+8010+7989.

FIG. 24, 25 or 26 showing a goniolens 7853 or 7800 laying on an eye, the treatment and diagnostic beams are coupled into the goniolens and eye via a final mirror 7995 or 8020. The mirror can be at least partially transmissible to the wavelength range that the digital camera is sensitive to, a digital camera with imaging optics 7853 being mounted behind that mirror such that the target region of the laser system is imaged through the goniolens, the mirror, the camera imaging optics 7895 and onto the digital camera sensor 7905. The camera unit 7853 housing can be connected or integrated to the mirror unit 8000, 8005 or 8010 but not to the goniolens unit 7801. Therefore, the goniolens unit can be mounted separate from the rest of the patient interface unit including the option to not be mounted at all and rather handheld via some mechanical feature such as a handle or fixed to the eye by a suction device.

In some embodiments, the integrated camera, mirror unit 8000, 8005 or 8010 can be mounted to the rest of the delivery system 7989. The camera and imaging optics unit 7853 can be integrated with the mirror unit either fixed or be adjustable in three dimensions, x, y and including focus z relative to the mirror unit to allow for its vision axis propagating through the mirror to be adjusted to become collinear to the treatment laser beam 7990 after being reflected by the mirror and propagating towards the eye and its focus z being calibrated to the treatment laser focus at some offset including zero. In this configuration, the patient interface unit up to and including the camera and mirror unit can be separately adjustable via system motion motors or by hand and the goniolens unit, being separately adjustable by hand/or through system motion motor that are movable independently from the rest of the patient interface. After target alignment, a laser treatment can be performed while the goniolens unit 7801 remains unconnected to the mirror unit 8000, 8005 or 8010.

In some embodiments, the patient interface unit can incorporate any of the patient interface features noted herein and further be configured such that the glass mounting structure is integrated with a digital camera and its imaging optics such that the camera and optics unit is fixed or adjustable relative to the mounting structure and that is aligned to image the target area once the glass has been connected or docked to the goniolens unit.

In some embodiments, the patient interface unit can incorporate any of the patient interface features noted herein and further be configured such that the glass mounting structure is integrated with a digital camera and its imaging optics such that the camera and optics unit is fixed or adjustable relative to the mounting structure and that is aligned to image the target area once the glass has been connected or docked to the goniolens unit.

In addition to or separate from the features discussed herein, the goniolens can comprise a camera that is integrated inside or near the goniolens. The camera can define a central visualization axis that can be slightly offset from the beam path of the treatment laser. Furthermore, the camera and its imaging optics can be very small, advantageously having a physical size in the two-dimension perpendicular to the imaging beam direction of <10 mm. Therefore, advantageously, the camera can be positioned in a direct optical path adjacent to or intermediate the beam paths created by the optic components discussed herein and the target area in the eye, without having to propagate through a beam combiner mirror. The treatment laser beam and other beams can therefore propagate next to the optical path of the camera advantageously through the main symmetrical axis of the goniolens and onto the target area of the eye to minimize aberrations for the treatment laser.

FIG. 17 illustrates an embodiment of a one-piece mirrored goniolens where a small camera and its imaging optics 7853, including optional illumination sources 7885, are integrated inside the goniolens or right behind the mirror to either allow direct imaging or an imaging path through the at least partially transmissible goniolens mirror 7891. If the mirror is placed at an angle where total internal reflection occurs for the imaging beam, therefore not allowing any beam part to transmit to the camera, then an optional coated prism 7890 can be installed right behind the mirror such that it makes an optical contact with the back side of the mirror and therefore allows some part of the imaging beam being transmitted through the mirror 7891, through the prism 7890 and into the camera unit 7853.

In accordance with some embodiments, the camera can be configured with the camera having a high sensitivity, therefore allowing to minimize the illumination level into the target area and still providing clear pictures and videos.

In accordance with some embodiments, the camera can be configured with the camera operating at an infrared wavelength or any other range that is not visible to the human eye. In particular, a wavelength range that does not trigger significant constriction of the pupil while the target area is illuminated by lighting in such a wavelength range.

In accordance with some embodiments, the camera can comprise one or multiple light sources 7885 with visible or infrared wavelength output, mounted separate or within the camera housing. The output of the light source(s) 7885 can be directed to illuminate the target area in the eye, and the power of the light source(s) 7885 can advantageously be adjustable in power and optionally in wavelength to modify the contrast of certain tissue layers in the target area that have different absorption and emission properties for different wavelengths of light. By changing the wavelength of the illumination source, different features of the target tissue area can be highlighted or suppressed, such as the pigmented and non-pigmented trabecular meshwork, Schwalbe's line 7600, the scleral spur 7915, the iris, an open backwall section of the Schlemm's canal and other anatomical features, some of which are illustrated in FIGS. 6, 7, 27-29. This will help the operator watching the camera output on a screen or an automated vision system connected or integrated with the laser system to get more information about the target tissue area of the patient's eye.

In accordance with some embodiments, the camera can comprise one or multiple light sources that comprise a LED, a fiber coupled led light source, a laser light source, and/or a fiber coupled laser light source.

In accordance with some embodiments, the camera can comprise an additional optional adjustable light source that is mounted anywhere in the delivery system and aligned such that it provides a light beam entering the goniolens in the central region such that the illumination propagated through the iris of the eye and terminating on the retina of the eye. This light source can be used to stimulate an iris contraction response in the patient's eye, where based on the adjusted power level of this light source and spot size on the retina, a stronger or weaker iris pupil closing effect can be achieved. In accordance with at least some embodiments disclosed herein is the realization that as the iris pupil closes more, the iris root close to the anterior angle structures gets pulled away from the angle and thereby opens up the angle more. This can allow for easier and greater access of the target tissue area in the anterior angle of the eye for the treatment laser, any diagnostic beam, any imaging and visualization beam and any illumination for the angle tissue. FIGS. 17, 20, 21, 22, 23, 24, 25 and 26 show such an embedded light source 7885 integrated in the patient interface along the central axis of the eye.

In accordance with some embodiments, the camera and its imaging optics can have a small physical size comparable or smaller than a camera and optics combination of a typical smart phone. The preferred size of the camera and optics combination can be <10 mm in each axis.

In accordance with some embodiments, the camera and its imaging optics can have a larger size such that its housing can be used as a handle to move the integrated camera goniolens unit. A preferred length of the camera housing 7920 length is >15 mm. Such embodiments are show in FIGS. 19 and 21 (see element 7920).

In accordance with some embodiments, the camera can comprise imaging optics that have a zoom capability to change the magnification of the image.

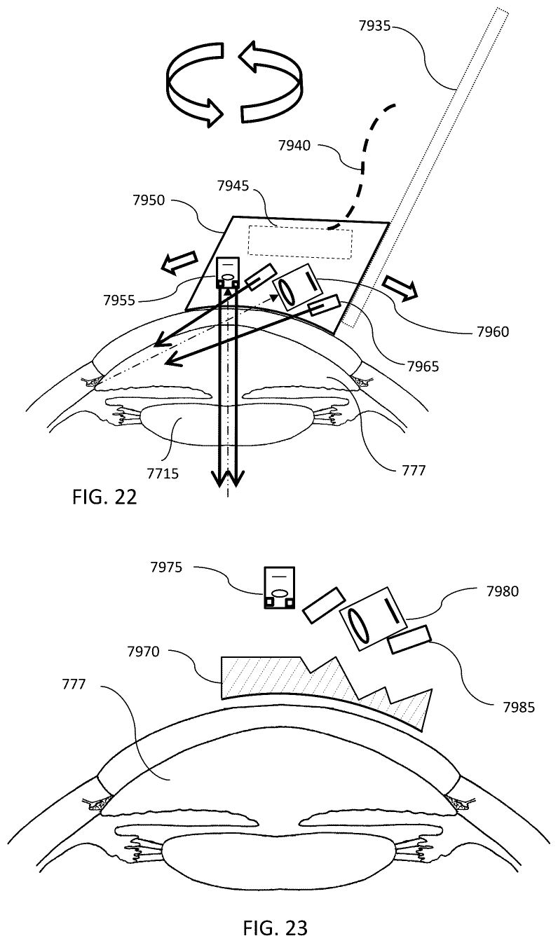

In accordance with some embodiments, the camera can comprise a camera output signal that is connected to a laser system computer via a cable or via a wireless connection and imaging data of this output signal can be used to verify and control system parameters before, during and after the treatment. FIG. 22 illustrates data 7940 that can be transferred from a wireless communication unit 7945. Optionally the wireless communication unit 7945 can further include a battery for power, electronic drivers and an embedded computer and software to control the cameras, illuminations and to process (and/or preprocess) and store the imaging data.

FIG. 24 illustrates a visualization system that utilizes a dual approach with a goniolens, a rotating mirror 7995, and an OCT imaging beam 7882 entering the limbus. Further, FIG. 25 illustrates a visualization system that utilizes a rotatable mirror unit 8005 above goniolens 7801 for 360° angle scanning. Furthermore, FIG. 26 illustrates a visualization system that utilizes a rotational mirror arrangement 8010, 8015 with central axis 8030.

As shown in FIGS. 24, 25 and 26, in accordance with some embodiments, the camera can comprise an additional focusing unit located between the goniolens and the laser routing mirror(s).

As shown in FIGS. 24, 25 and 26, in accordance with some embodiments, the camera can be configured such that one or more laser routing mirrors comprises a curved mirror, for improving the focusing and aberration control of the delivery system.

As also shown in FIGS. 24, 25 and 26, in some embodiments, the camera can be configured such that one or more laser routing mirrors comprises a mechanically tiltable mirror, for enhancing or replacing the scanning systems of the delivery system.

Standalone Goniolens with Integrated Camera

In accordance with some embodiments, there is provided a standalone goniolens with an integrated digital camera and imaging optics, referred to from here on as a camera-goniolens unit.

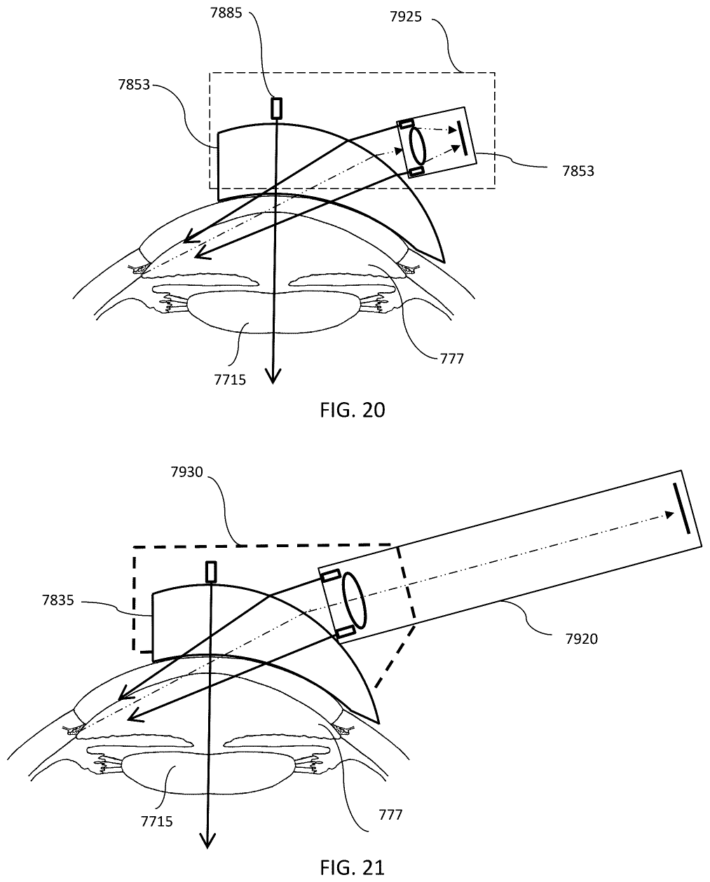

In some embodiments, the camera-goniolens unit can comprise a direct goniolens without any mirror and having its camera either integrated within the optical goniolens substrate FIG. 22 (camera units 7960 and 7955 and illumination units 7965 and in 7955) or being mounted next to the goniolens substrate FIG. 19 (see element 7915), FIG. 20 (see element 7925), FIG. 21 (see element 7930) or FIG. 24 (see element 8000). The camera can have an alignment that provides an image of the anterior angle of the eye when the unit is placed on the eye. FIG. 20 is a side cross-sectional view of an eye illustrating a visualization system that utilizes a rotating camera-lens assembly 7853 positioned over the cornea. FIG. 21 is a side cross-sectional view of an eye illustrating a visualization system that utilizes a handheld camera-lens block 7930 with a limbus cutout 7835. FIG. 22 is a side cross-sectional view of an eye illustrating a visualization system that utilizes a combined camera-goniolens unit 7950 with rotating handle and one or more imaging subunits 7945. A second optional camera is placed at location that allows a straight down view to the retina area of the eye for retinal imaging of the eye. FIG. 22 (see element 7955).

FIG. 22 is a side cross-sectional view of an eye illustrating a visualization system that utilizes a combined camera-goniolens unit 7950 with a rotating handle and at least one imaging subunit 7945. FIG. 23 illustrates a visualization system comprising one or more integrated camera-lens submodules 7970, 7980 in an exploded view, in accordance with some embodiments. As shown, in one embodiment the camera-goniolens unit 7950 can comprise a direct goniolens without any mirror and having its camera integrated within the optical goniolens substrate.

FIG. 22 shows the integrated camera units 7960 for anterior angle visualization, camera unit 7955 for iris, lens 7715 in the eye and retina visualization, and illumination units 7965 and inside or next to 7955. An optional handle 7935 or grabbing features on the unit 7950 can allow this unit to be moved on the eye. Advantageously, the combined camera-goniolens unit 7950 can be rotated to image all parts of the 360 deg angle tissue in the eye, as well as being moved up and down and sideways on the cornea to adjust the lateral view position of the central camera 7955 as well as the visualization approach of camera 7960 into the irido-corneal angle region of the eye.

FIG. 23 shows an exploded view of FIG. 22 wherein all camera and illumination units float above their integrated positions over the specifically shaped goniolens 7970 that contains a standard concave bottom surface and an irregular shaped upper surface that is shaped such that it provides aligned window surfaces that can accept the individual modules so that their aiming direction into the eye may be preset and prealigned. The goniolens 7970 shape is specifically manufactured to accommodate a specific set of camera and illumination units that are selected to be integrated in this unit.

In another embodiment the camera-goniolens can comprise a mirrored goniolens and having its camera either integrated within the optical goniolens substrate or being mounted next to the goniolens substrate and in this case the optical path of the camera entering the goniolens mirror from above.

In some embodiments, the combined camera-goniolens unit 7950 can comprise an additional holding stick or grip features integrated in a housing such that the unit can be held and manipulated by hand.

In some embodiments, the combined camera-goniolens unit 7950 can comprise an additional mounting flange with an integrated suction ring to allow the camera-goniolens unit to be held in place on the eye without the operator having to hold it with a hand.

In some embodiments, the combined camera-goniolens unit 7950 can be configured such that the camera and imaging unit is a small unit <10 mm in all 3 dimensions or at least 2 of 3 dimensions.

In some embodiments, the combined camera-goniolens unit 7950 can be configured such that the camera and imaging unit is a larger unit >10 mm in length and/or includes a zoom lens. The body of the camera housing can be optionally used as a handle to manipulate, move and adjust the camera-goniolens unit on the eye. See, for example, FIG. 19 and FIG. 21 or FIG. 25, wherein a large camera 7920 is integrated above the mirror 7995.

In some embodiments, the combined camera-goniolens unit 7950 can be configured such that the camera signal is transmitted to a display or a computer system of any other imaging, diagnostics, or treatment system through a electrical cable.

In some embodiments, the combined camera-goniolens unit 7950 can be configured such that the camera pictures and videos are recorded on or to a memory device that is integrated in the standalone camera-goniolens unit.

In some embodiments, the combined camera-goniolens unit 7950 can be configured such that the camera signal is transmitted to a display or a computer system of any other imaging, diagnostics, or treatment system through a wireless transmission and/or such that the unit 7950 is powered by a integrated battery.

The combined camera-goniolens unit 7950 can also comprise one or multiple light sources with visible or infrared wavelength output that is mounted separate or within the camera housing. The output of the light source(s) can be directed to illuminate the target area in the eye and its power can advantageously be adjustable in power and optionally in wavelength to modify the contrast of certain tissue layers in the target area that have different absorption and emission properties for different wavelengths of light. By changing the wavelength of the illumination source, different features of the target tissue area can be highlighted or suppressed, such as the pigmented and non-pigmented trabecular meshwork, Schwalbe's line, the scleral spur, the iris, an open backwall section of the Schlemm's canal and other anatomical features, as described herein. This will help the operator watching the camera output on a screen or an automated vision system connected or integrated with another system to get more information about the angle tissue area of an eye.

The combined camera-goniolens unit 7950 can also comprise one or multiple light sources, as described above, wherein the preferred light source is either a LED source, a fiber-coupled LED light source, a laser light source, or a fiber-coupled laser light source.

The combined camera-goniolens unit 7950 can also comprise an additional optional adjustable light source that is mounted and aligned that it provides a light beam entering the goniolens in the central region, as shown in FIG. 22 as element 7955. This can permit illumination to be propagated through the iris of the eye and terminate on the retina of the eye. This light source can be used to stimulate an iris contraction response in the patient's eye, wherein based on the adjusted power level of this light source and spot size on the retina. In this manner, a stronger or weaker iris pupil closing effect can be achieved. As the iris pupil closes more, the iris base close to the anterior angle gets pulled away from the angle and thereby opens up the angle more. This allows for easier and greater visibility in the anterior angle of the eye. FIG. 22 (within 7955) shows such a light source integrated in the goniolens camera unit 7955.

The combined camera-goniolens unit 7950 can also be configured such that the unit is part of a patient interface that is either permanently connected to a laser delivery system or detachable and dockable to a laser delivery system. FIGS. 17, 24, 25 and 26 show the here-embodied versions that provide a dockable or permanent mechanical connection to a laser delivery system 7989.

The combined camera-goniolens unit 7950 can also be configured such that the gonio lens part allows a clear path for a treatment and or diagnostic laser through the goniolens and into the eye, while the camera and illumination units within this goniolens are shifted out of the central path of the gonio lens such that the treatment and/or diagnostic beam can propagate through the gonio lens and into the eye without clipping or being obstructed by the camera and illumination units.

In accordance with some embodiments, the combined camera-goniolens unit 7950 in FIG. 26 illustrates that a laser input beam can enter centered and parallel to the main symmetry axis of the eye and therefore allow access to all 360 degrees of irido-corneal angle tissue of the eye by rotating the two-mirror assembly 8010 around this center axis. In some embodiments, the mechanical connection of the entire connected unit 8010+7801 can connect to the rest of the laser delivery system above 7989 via rotatable joint. In some embodiments, an additional rotatable interface joint can be present between the two-mirror unit 8010 and the goniolens unit 7801, therefore allowing the goniolens to remain stationary on the eye and fixed via a suction device, while the rest of the patient interface above rotates to allow full 360 deg access to the anterior angle of the eye. Other than the rotational joints all units 7801+8010+7989 can be fixedly connected and no docking procedure need be performed. The entire combined unit 7801+8010+7989 can advantageously be moved and placed on the eye by hand and then is held in place during the treatment procedure either by hand/or via a suction device.

The combined camera-goniolens unit 7950 can also be configured to provide a cutout, as discussed herein, wherein one side of the goniolens is cut off as in FIGS. 16, 19, 20, 21, 22 and 24. This can open up access to the limbus area and part of the cornea over the target zone that is defined by the laser or imaging beams entering the goniolens from the other side. These cutout versions can be used in a system that contains diagnostic, imaging and/or treatment beams that enters the limbus area over the target area from above as described below. See FIG. 16 or 24 for examples of such features for the combined camera-goniolens unit 7950.

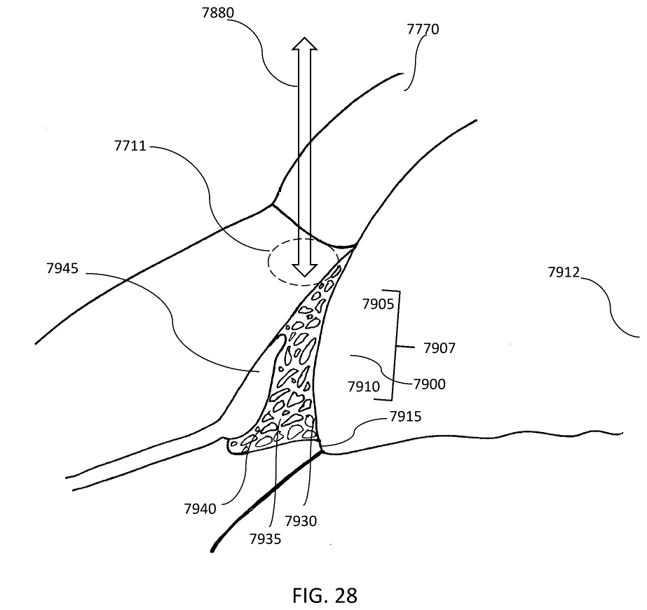

As shown in FIG. 24, some embodiments of the system can be configured such that the target area at the anterior angle area of the patient's eye can be treated via a treatment laser beam 7990 coming through a goniolens 7853 and therefore entering the cornea of the eye on the opposite side from the target area 7725. In some embodiments, the combined camera-goniolens unit 7950 can be used to provide pre-operative care, can be used during treatment, and can be used during post-op diagnostics and imaging of the target area 7725 by penetrating the tissue layers in the area right above the target area with a one or multiple diagnostic and imaging beams 7880. The tissue layers above the target area are around the limbus 7705 and consist of sclera tissue 7710, conjunctival tissue and outer cornea tissue layers in 7770, as shown in FIG. 28. In accordance with an aspect of at least some embodiments disclosed herein is the realization that the tissue layers other than the cornea tissue cannot be easily penetrated by light sources including lasers. Particularly the scleral tissue layers 7710 can cause a large amount of light scattering, absorption and aberrations. The amount of beam degradation because of scattering, absorption and aberrations is wavelength dependent. The presently disclosed systems 7882, such as in FIG. 24, can be used to penetrate this tissue area of the eye and provide useful diagnostics and imaging data. In some embodiments, the system can utilize OCT to penetrate scattering and absorbing tissue layers.

For example, in some embodiments, an OCT beam 7880 can be used to penetrate the tissue layers entering the eye anywhere between 5 mm up from the limbus into the cornea and 10 mm down from the limbus into the sclera from here on referred to as the entry area. The OCT beam enters the eye at an angle that is parallel to the main optical axis of the eye or tilted from this vertical axis by up to 60 degrees to either side. This OCT imaging beam provides data that allows for visualization of the tissue layers in the target region of the eye, including the outline, size and location of the layers of trabecular meshwork 7907, Schlemm's canal 7945 and other tissue layers. This data is then used by the operator to assess the target area 7725 and prepare the treatment parameters. In one embodiment this data is used to assess the shape and integrity of Schlemm's canal 7945.

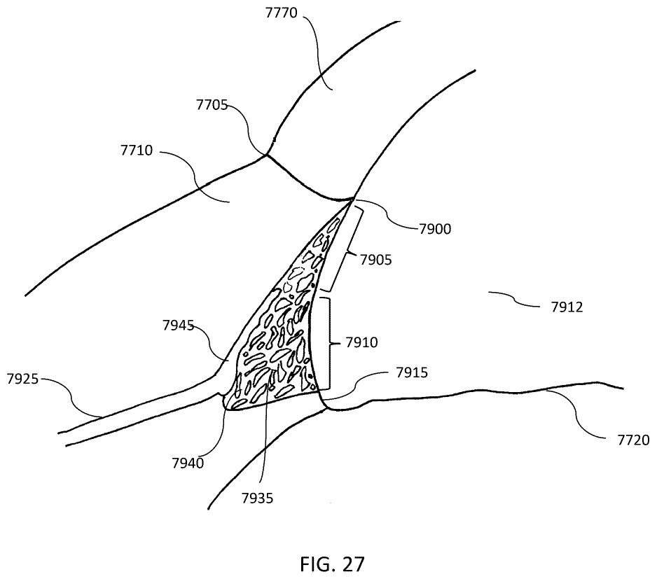

FIG. 27 is a side cross-sectional view of a trabecular meshwork of an eye having a collapsed Schlemm's canal 7945 and meshwork layers 7905, 7910. Further, FIG. 28 is a side cross-sectional view of a trabecular meshwork of an eye illustrating another angle cross-section for scanning 7880 over a collapsed or partially open Schlemm's canal.

As demonstrated in FIG. 28 showing a healthy eye or an eye in an early glaucoma stage the Schlemm's canal width in the radial axis of the eye is much larger than in an eye that has more tissue degradation due to more advance glaucoma and has a partially collapsed Schlemm's canal 7945, as shown in FIG. 27. This information is optionally used to adjust the laser treatment parameter. Furthermore, the OCT data is used to calibrate and guide the laser treatment in the target area either by a fully automated image analyzation system that adjusts and sets the system parameters before and during the treatment procedure, or by providing the data displayed on a monitor, such that the operator can make system parameter adjustments based on the measured OCT data or a combination of both.

Based on the OCT beam location through the limbus area the quality of the OCT imaging data will vary. For example, by aligning the OCT beam to enter in the region of the transition zone between the cornea and the sclera, see FIG. 28, 7880 the OCT beam 7880 has an easier and higher penetration ability since less of the scattering tissue 7711 (Sclera starting part) is in its beam path. This allows for easy detection of particularly the upper regions of the trabecular meshwork 7905. And that data is then used to calibrate the system parameters including monitoring this data live during the laser treatment and making laser system adjustment during the treatment to accommodate or compensate any movement or changes of the treated tissue layers.

In some embodiments, as shown in FIG. 24, the OCT beam 7880 can propagate into the eye via the entry area of a cutout, without any optical interface connected to the eye, therefore enter the eye tissue layers from the air.

In some embodiments, as shown in FIG. 24, a glass or clear material substrate 7883 can be placed over at least part of the entry area such that the OCT beam 7880 is propagating into the entry area of the eye via the substrate.

In some embodiments, the OCT imaging system can be integrated into the laser treatment system in a way that it shares at least a control system part or a user interface part or a delivery system part or any combination thereof.

In some embodiments, as shown in FIG. 24, the OCT imaging system unit 7882 can be mechanically connected to a patient interface, as shown via the mounting structure 8000 in FIG. 24. The mounting structure 8000 can be configured to allow the OCT beam to enter the eye through the entry area while simultaneously allowing the treatment laser beam 7990 to enter the eye 777 through the goniolens 7853 around the opposite side of the cornea. In some embodiments, the mounting structure 8000 can comprise one or more mirror components.

FIG. 24 also illustrates a dual patient interface that can comprise at least a goniolens that is mechanically mounted to a housing part or mounting structure 8000 of the patient interface. The system can contact the eye and allows a treatment beam 7990 to enter the eye from one side and having a cutout or open access area at the limbus area referred to earlier as the entry area on the side opposite to the treatment laser entry of the eye. This housing cutout opening in the patient interface, can be positioned above the treatment target area of the eye and enable an OCT imaging beam to enter the eye through an optional substrate 7883 that lays on the limbus area and that can be optionally integrated and mechanically connected to the housing 8000.

In some embodiments, the dual patient interface can be configured such that the mechanical housing 8000 is further connected to at least part of the OCT imaging beam delivery system optics, part of 7882.

In some embodiments, the OCT imaging beam can be part of a standalone diagnostic system that is used independent from any treatment system and optionally also independent from any goniolens imaging unit. In some embodiments, the standalone system can comprise a scanning system that scans the OCT beam in addition to the target tissue area also around at least part or all of the circumference of the entire circular limbus area of a patient's eye to create imaging data of part or all of the eye's anterior angle area.

In some embodiments, an imaging system is provided in which instead of OCT, a two-photon microscopy detection system or a second harmony detection system or a combination of these detection systems is used.

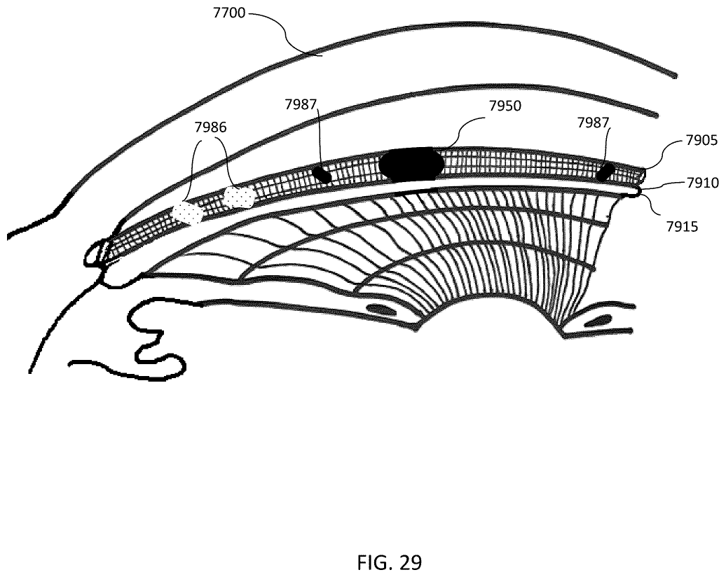

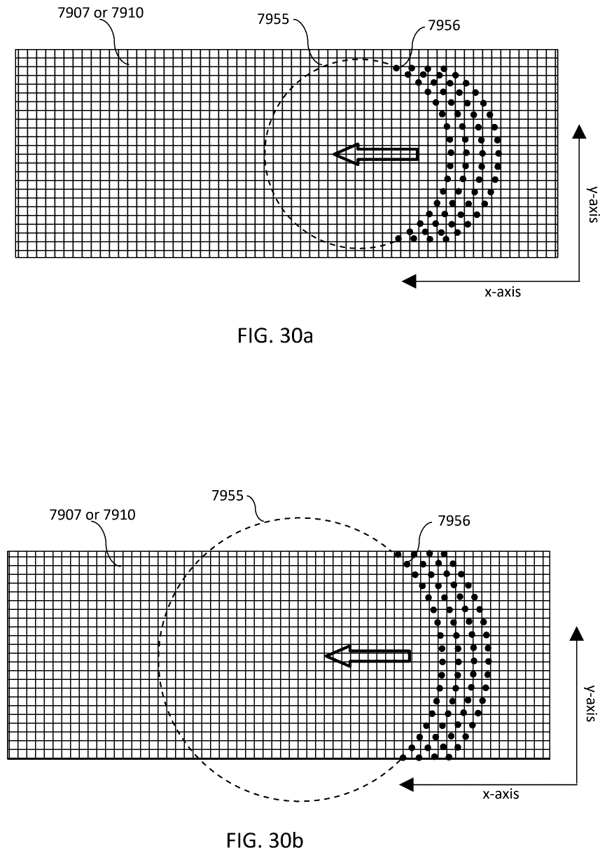

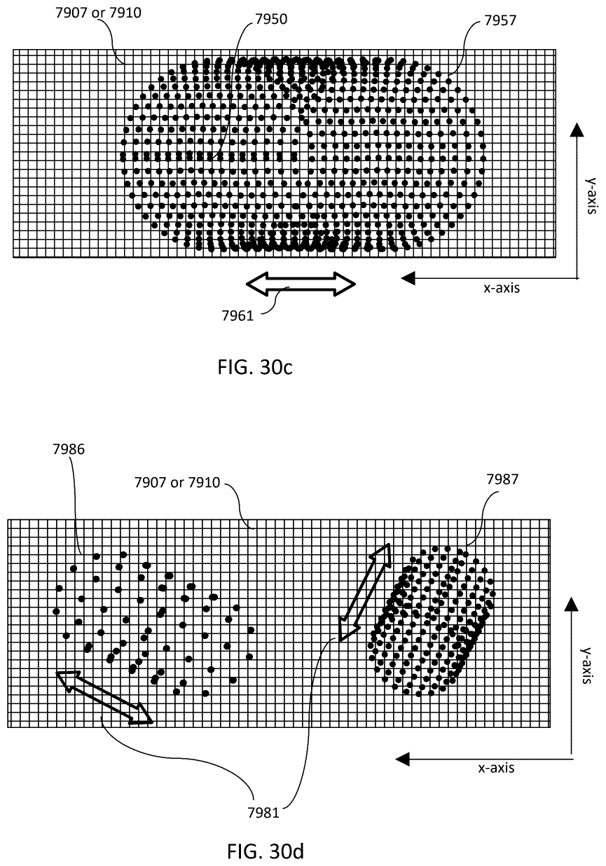

FIG. 29 is a partial top view of an anterior angle of an eye with a target zone 7950, illustrating different laser treatment scanning pattern methods in an anterior angle tissue target area of an eye. In particular, the scanning pattern method can treat a target area that comprises at least part of the trabecular meshwork 7910 and 7905. The scanning pattern method can be used to create a large hole 7950 through the trabecular meshwork or a small hole 2x 7987 or a “Soft-Hole” see [211], 2x 7986 and thereby allowing for aqueous outflow from the anterior chamber into Schlemm's canal 7945 for reducing the IOP of the eye and thereby treat glaucoma. FIGS. 30a, 30b, 30c and 30d are a magnified view of the Trabecular Meshwork sections here in FIG. 29 and illustrate various aspects of laser scanning patterns disclosed herein. The scanning pattern here disclosed are not limited to the Trabecular Meshwork but can also be applied to any other tissue target area in an eye, such as lens capsule, retina, vitreous floaters, iris, cornea, scleral spur and others.

For example, FIG. 30a is a plan view of a circular scanning pattern with arcs 7956 over a grid representing a portion of the trabecular meshwork 7907, 7910, in accordance with some embodiments. The trabecular meshwork illustrated can represent a portion of the entire meshwork 7907 or a portion of the pigmented trabecular meshwork portion 7910. The treatment laser focus is here being scanned in a circular motion represented by dashed lines 7955, while the resultant scanning circle is at the same time being scanned from the right to the left (large arrow), thus creating a series of overlapping scan loops.

In FIGS. 30a-30c, the dots 7956 represent laser shots that have a spot separation of about 3 um to about 100 um. The spot separation can be between about 2 um to about 60 um, about 4 um to about 40 um, about 6 um to about 20 um, or about 8 um to about 10 um. The laser shots 7956 can be scanned in a scan circle or loop with a diameter of between about 20 um to about 400 um, about 50 um to about 300 um, about 80 um to about 200 um, about 100 um to about 150 um, or about 180 um, or about 50 um, about 80 um, about 200 um, or about 250 um. Thus, the laser shots 7956 can be distributed in a partial circle or arc (see FIGS. 30a and 30b) or in a full circle or loop (see FIG. 30c), in a variety of spot sizes and arc/loop diameters.

In the scanning method for FIGS. 30a and 30b, the laser spot can be scanned on a circular path, but the laser may be disabled for most of this circle 7955 and only enabled during the circle arc that forms the series of dots 7956 on the right. The translation of the circle to the left (x-axis) is continued until the desired cross section of hole size in the x-axis is achieved. After the circle arcs have been scanned from the right to the left here along the x-axis the cross section of the hole that is being created is equal to the vertical arc size in the y-axis and horizontal scanning length in the x-axis.

Typical hole size values are for the x-axis may be between about 20 um to about 1000 um, about 50 um to about 100 um, about 200 um to about 600 um, about 300 um to about 500 um, or about 40 um. Typical hole size values are for the y-axis may be between about 10 um to about 500 um, about 50 um to about 200 um, about 80 um to about 300 um, about 40 um to about 200 um, or about 100 um.

After the circular arc sideways scan along the x-axis has been completed, the z-axis depth of the cut is changed, and the circular arc cutting pattern is repeated again along the x-axis in a different plane along the z-axis.

In some embodiments, the z-axis scanner (e.g. vertical double arrow in 7360, FIG. 18b) is kept at a fixed depth while the circle arcs are cutting from left to right (or right to left) and then the z-axis is adjusted more posterior towards Schlemm's canal or more anterior towards the central eye axis 8030. Depending on which direction the z-scan is being performed (inside out or outside in).

Further, in some embodiments, the z-scan can be performed during the arc scan pattern and thereby create an arc cut layer that is ramped up or down in the z-axis (depending on whether the z-scan is performed inside out or outside in), in either a linearly or a curvilinearly z-ramp. Typical z-axis layer separation (for either parallel z-layers or ramped up or down z-layers) is set to be between about 7 um and about 100 um, about 10 um and about 150 um, about 15 um and about 100 um, or about 20 um and about 50 um, based on treatment area of the trabecular meshwork (where the thickness is generally between 50-80 μm in the anterior region and between 100-130 μm in the posterior region). This means that a full hole through the trabecular meshwork to open up Schlemm's canal can comprise between about 3 to about 100, about 10 to about 70, about 20 to about 50, or about 30 to about 40, z-axis layers of arc cuts on top of each other (either parallel or in a ramp-like structure).

In accordance with some embodiments, FIG. 30b illustrates that the circular scan diameter can be larger than the vertical dimension (y-axis) of the target area in the y-direction. The arcs where the laser pulses are “on” or “fired” can be small enough that they fit inside the target y-area. This allows the cutting dimension in the y-axis to be smaller than the circular diameter of the circular scan. It also makes the cutting arcs less curved compared to FIG. 30a. Such embodiments of the scanning and firing procedure can advantageously facilitate tissue disruption in a more stepwise fashion, which may be desirable for certain anatomies, patients, and treatment areas. Otherwise, all scanning methods from FIG. 30a apply here also to FIG. 30b.

In some embodiments, as shown in FIG. 30c, the laser pulses may be scanned continuously throughout a pattern with a certain spot separation depending on the laser repetition rate and the scanning speed or the laser may be continuously on (CW-Laser). Therefore, the laser can form cuts in full circular or capsule shaped scanning patterns while being translated in the x-axis (right to left or left to right or both repeatedly). This results in an inhomogeneous laser spot placement (or treatment) density, which becomes irrelevant if photo-disruptive laser pulses are scanned with sufficient spot energy and density to remove the target material and thereby creating a hole (removing the tissue in the target layer) or otherwise may be desirable for certain anatomies, patients, and treatment areas.

As can be seen in FIG. 30c, the upper and lower middle sections receive a higher laser spot density than the central regions. This inhomogeneous spot density placement has no significant downside because the goal is to remove the scanned tissue sections and therefore a higher spot density or “overshooting” in certain areas does not reduce the ability to remove material. The advantage of this scanning pattern is that the laser complexity is reduced because no rapid laser on and off switching means needs to be incorporated within a circular scan. However, one minor downside is that the overall deposited laser energy may be somewhat higher than required. All other scanning method aspects (e.g. the z-axis scan) are identical as described for FIG. 30a.

In some embodiments, a full-circle or full-loop scan and firing pattern can be performed wherein the laser intermittently fires as it is scanned around the looped scanning pattern. Intermittent scanning can enable the spot density to be tuned in order to avoid areas of overshooting or relatively higher spot density. Moreover, exemplary scanning patterns, optical systems, rotating optics, and related methods are also discussed and illustrated in copending U.S. Pat. App. No. 63/750,772, filed on Jan. 29, 2025, the entirety of which is incorporated herein by reference.

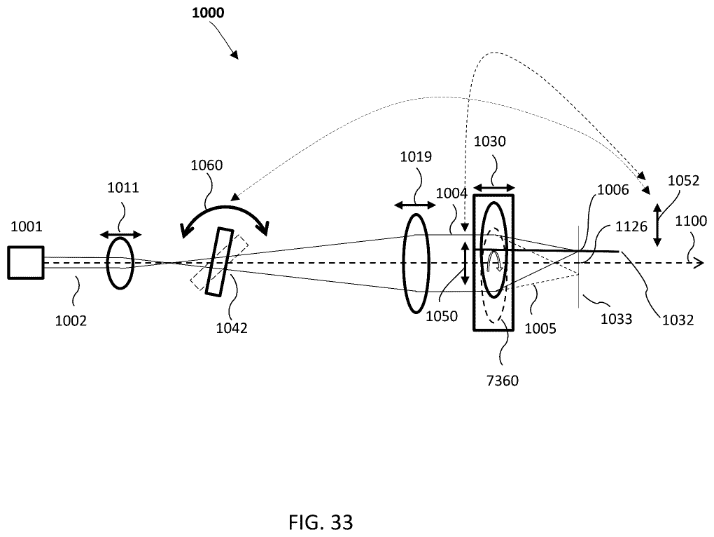

Some embodiments of the methods discussed herein can form the circular or capsule shaped scanning patterns via the unit 7350, whereby the scanning system and the treatment laser beam 7990 is scanned by spinning and/or translating one or more focusing optics or components of a focusing optics assembly FIG. 18b or FIG. 33, 7360, thereby creating a circular scan pattern. The spinning of the beam FIG. 33, 1005, which is passing through the offset 1032 rotating optic(s) 7360, thereby enables a circular pattern 1126 to be created. The focusing optic can be in the form of a single lens, a multi-lens such as an achromat, a aspherical lens or any combination thereof that is designed to control the aberrations and allows the performance to reach the desired laser spot size. Additional lateral (x-y) scanning of the circular scan is achieved by translating FIG. 33, 1050 the focusing optics 7360 perpendicular to the optical axis 1100 or by adjusting the rotational angle 1060 of a parallel tilted glass plate/window FIG. 33, 1042 placed inside a telescope defined by lens 1011 and lens 1019, or doing both translation methods together (e.g. one in the x-axis and one in the y-axis). This combination of circular scan plus a translation scan is here referred to as a capsule shaped scanning pattern. Finally, translating either the focusing lens assembly 1030 or any of the telescope lenses 1011 or 1019 backwards and forwards along the optical axis 1100 adds the z-axis depth scanning ability to the scan pattern and thereby achieves full 3D scanning in the target volume. It is understood that alle translation motions and rotation motions are driven by motors such as stepper, servo, linear or any other form of motor and such motors being controlled by the control system 7050.

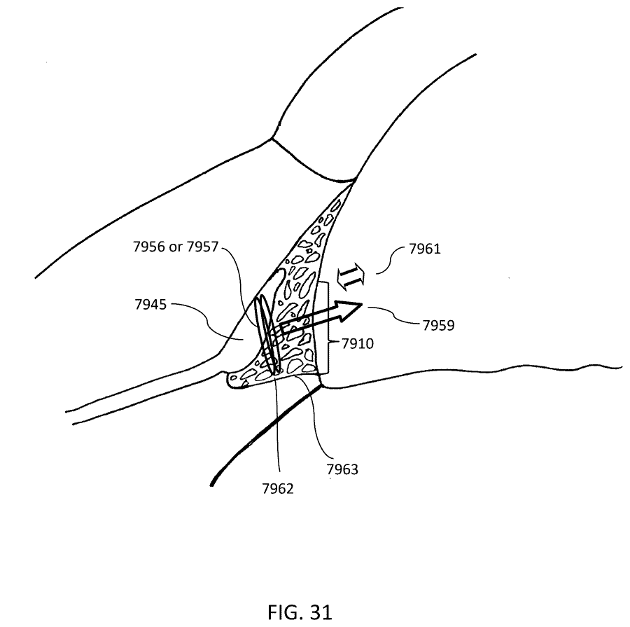

Thus, the treatment methods of FIGS. 30a-30c and FIG. 30d right side pattern can be used to treat a trabecular meshwork of an eye in a pattern targeting meshwork layers with showing a one scan layer (partial thickness) cut in the z-axis 7959 along a scanning axis 7961 (here representing the x-axis of FIG. 30a-30c along the circumference of the Trabecular Meshwork, coming out of the views plane here), as shown in FIG. 31.

FIG. 30d illustrates another treatment procedure with the scan pattern on the left 7986. This capsule shaped scan pattern has a low laser spot density and therefore the laser will not create a complete tissue removal of this plane. This low density photo disruptive laser spots pattern results in a “Soft-Hole” creation where a few micro holes are created around a small area of each laser spot, but the majority of the Trabecular Meshwork tissue remains untouched. This leads to a revitalization/rejuvenation effect of the Trabecular Meshwork tissue and increased aqueous humor outflow due to the micros holes without creating a more tissue disrupting full home as shown with the high density pattern on the right 7987.

Further, FIG. 31 also shows a side view of the scanning method described in FIG. 30c with the first scan plane in the z-axis starting in or near Schlemm's canal 7962 and then scanning upwards towards the center of the eye 7959 while scanning in circles 7957 and left and/or right in the x-axis in the direction of arrow 7961. This illustrates an “inside-out cut,” starting the spots in or near Schlemm's canal and then successively working its way up through the trabecular meshwork 7910 and thereby creating a hole in the trabecular meshwork. The z-cut direction can also be reversed to perform a “outside-in” cut.

In some embodiments, the starting plane in the z-axis 7963 can be fully within the trabecular meshwork and the z-scanning direction can also be upwards as indicated by the direction arrow 7959. This process can result in a thinning of the trabecular meshwork by removing some of the anterior side (towards the center of the eye) of the trabecular meshwork. Although this does not result in a full opening through the trabecular meshwork, the remaining partial thickness meshwork layers advantageously now have an increased outflow capacity and therefore also result in the IOP reduction of the eye.

One of the significant advantages of this partial removal is that the inner transition layer of the trabecular meshwork and the inner layers of Schlemm's canal are never disrupted and this leads to a lower tissue reaction, inflammation, fibrosis during the healing and remodeling phase which results in less post op outflow resistance built up through these healing processes. For such a partial thickness trabecular meshwork cut, a larger x-axis dimension may be beneficial to create a more substantial effect. Also advantageously and unlike any conventional treatment method, such a partial thickness cut can be performed at up to 360 degrees around the entire trabecular meshwork in a non-invasive procedure.

In some embodiments, this partial thickness cut can also be performed from the top layers of the trabecular meshwork towards the final layer at the depth 7963, meaning from outside to inside in the z-axis.

The circular scanning patterns as described for FIGS. 30a, 30b and 30c can be created with a circular focusing lens scanning system as described in FIG. 18b and FIG. 32. FIG. 32 is a schematic view of a beam-intensity profile 520 and scanning lens offset method 182 to perform a scanning procedure.

### Optical System Features

FIG. 33 is a schematic view of an optical system and optical components thereof that collectively operate to achieve a circular or capsule shaped scanning pattern. FIG. 33 shows an optical system 1000 that can comprise a beam expanding telescope having a rotating optical element, such as a lens, parallel plate, or wedge. Other optical elements (e.g., lenses, parallel plates, or wedges) can be used in the optical system 1000 for collimating, focusing, offsetting, and/or translating a laser beam passing through the optical system. In operation, the laser beam can approach the rotating optical element along a pre-rotation beam axis, and after passing through the optical element, the laser beam will be offset from the pre-rotation beam axis to an offset beam axis. Further, due to the rotation of the optical element, the offset beam axis can also rotate (generally about a “projection” of the pre-rotation beam axis) to create a circular pattern of the laser's focus point on a focal plane of the laser.

For example, in the embodiment shown in FIG. 33, the optical system 1000 can comprise one or more lenses 1011 and 1019 (such as telescope lenses) that are disposed along a system optical axis 1100 of the optical system 1000, a focusing lens 7360 that is mounted in a lens rotation assembly 1030 and disposed along the system optical axis 1100, and a tiltable optic 1042. The focusing lens 7360 can have a central lens optical axis 1032 that is offset from a rotation axis of the lens rotation assembly 1030 (and the axis of the lens rotation assembly can be colinear with the system optical axis 1100).

In operation, the laser beam 1002 passes through the lens 7360 as the lens 1031 rotates, whereby a focus point 1006 of the laser will move in a circular pattern 1126 in the focal plane 1033. The shape of the pattern 1026 may be a circle having a diameter that is equal to two times the lens offset.

In accordance with some embodiments, the input diameter 1004 of the laser beam 1002 as it enters the into the rotating focusing lens 7360 can be small enough relative to the focusing lens diameter such that the entire beam always falls onto the focusing lens without any “clipping” as the focusing lens 7360 rotates and/or translates/moves relative to the stationary and approximately collimated input beam 1004. For example, the input diameter 1004 can be less than (or no greater than) two times the difference of the radius of the focusing lens 7360 minus the offset of the focusing lens 7360.

Further, in accordance with some embodiments, z-axis control for changing the focal plane 1033 to achieve volumetric scanning can be achieved by translating either of the telescope lenses 1011 or 1019 or the lens rotation assembly 1030 along the optical axis 1100, as indicated by the arrows above the lenses 1011, 1019, 7360.

Furthermore, a sideways translation and/or tilting or reciprocal rotation of one or more of the optical components of the optical system 1000 can be performed to create a sideways x-axis (or y-axis) translation of the scan, as shown and discussed with regard to FIGS. 30a-30c (note the x-axis translation arrows, such as arrow 7961 in FIG. 30c). In this manner, the laser beam can approach a translatable/tiltable optic of the system along a pre-tilt beam axis and be moved or translated (linearly or curvilinearly) to an offset, tilted axis, which tilted axis can move along the x-axis for providing translation of the laser focus point across the focal plane. In some embodiments, the translatable/tiltable optic can be immediately prior to the lens rotation assembly 1030, such that the tilted axis of the laser beam is also defined as the pre-rotation beam axis, as shown in FIG. 33.

For example, in some embodiments, the sideways translation of the scan can be performed by translating the lens rotation assembly 1030 in a direction transverse or perpendicular relative to the system optical axis 1100 (and in some embodiments, the direction of translation can be in a direction that passes through the system optical axis 1100), as indicated by the vertical arrow 1050 to the left of the lens rotation assembly 1030 in FIG. 33.

Additionally or alternatively, some embodiments can be provided in which a sideways translation of the scan can move laterally (perpendicular to the system optical axis 1100) by tilting or reciprocally rotating one or more of the optical components of the optical system 1000 about an axis that is oriented transverse or perpendicular relative to the system optical axis 1100.

For example, the optical system 1000 can comprise a tiltable optic or window 1042. The tiltable optic 1042 can be placed inside the telescope and be rotated 1060 around an axis that is oriented transverse relative to the system optical axis 1100 (and optionally passes through the optical axis 1100 and/or is oriented perpendicular relative to the system optical axis 1100). The tilting or rotation 1060 of the tiltable optic 1042 can create a beam shift and tilt that results in a translation 1052 (shown as an arrow in the x or y-axial direction) of the focus point 1006 across the focal plan 1033. The resultant translation can create a linear or curvilinear translation of the center of the circular pattern 1126 across the focal plane 1033, as desired.

Thus, the center of the circular pattern 1126 can be shifted by translating the lens rotation assembly 1030 and/or tilting or reciprocally rotating an optical component of the system 1000 about an axis that is transverse or perpendicular relative to the system optical axis 1100.

For the embodiment where this scanning system is used to propagate the focusing beam FIG. 33, 1005 through a goniolens and into an eye to treat areas of the Trabecular Meshwork. The axis that represents the 360 degree circumference path of the Trabecular Meshwork has been labeled x-axis in FIG. 30a-30d and FIG. 31. However as the delivery system laser beam is targeted around the 360 deg circumference of the Trabecular Meshwork (e.g. by rotating a mirrored goniolens), the association between the delivery system x-axis and the Trabecular Meshwork x-axis is due to the rotation no longer synchronized. For example, if the system is aligned such that a translation in the x-axis of the delivery system (e.g. translating 1050 the focusing lens assembly 1030 as shown in FIG. 33) corresponds to the x-axis of the Trabecular Meshwork as shown in FIG. 30a-30d, then rotating the mirrored goniolens by 90 degrees such that the Trabecular Meshwork target area now is in a 90 deg rotated segment of the eye, will result in a translation scanning in the eye in the y-axis (along the “height” of the Trabecular Meshwork). To compensate this rotation and keeping the axial reference synchronized, the translation 1050 or 1060 can be expanded to a two dimensional translation (rotation) and then can be continuously adjusted to gradually add in a y-axis component such that the resulting translation (of the main long axis of the capsule pattern shape) in the eye always remains along the circumference axis of the trabecular Meshwork. Though the preferred embodiment here is a low complexity solution, where the circular scan diameter is made smaller (between 10 μm and 200 μm) and the translation 1050 or 1060 of the delivery system remains one dimensional and at a random angle relative to the Trabecular Meshwork axis. To fully fill a scanning area e.g. FIG. 30c without leaving a middle gap void of laser spots, the minimum translation distance is equal to one scan circle diameter. Since the translation axis is now transformed to a random lateral axis on the Trabecular Meshwork, the resulting capsule pattern will be anywhere between horizontal to vertical as the example shown in the two patterns presented in FIG. 30d. The effective translation axis here are indicated by 7981. Using for example a preferred small scan circle diameter of around 50 μm will result in a minimum capsule shape size of 50 μm×100 μm. See right pattern in FIG. 30d, 7987, showing a high density laser pattern tilted mostly in the y-axis. Such a small high density laser spot pattern is ideal (when combined with a z-scan) to cut a hole through the Trabecular Meshwork. That fact that the main axis of this capsule pattern is maybe randomly oriented plays no role since it will entirely fit within the Trabecular Meshwork height at any translation scan (main long capsule axis) angle. Having to reduce the size of the hole is also no limitation since outflow calculations for a typical eye demonstrate that even a circular hole of around 30 μm will be able to allows the entire aqueous humor production of an eye to flow through such a 30 μm Trabecular Meshwork hole at an IOP of less than 10 mmHg.

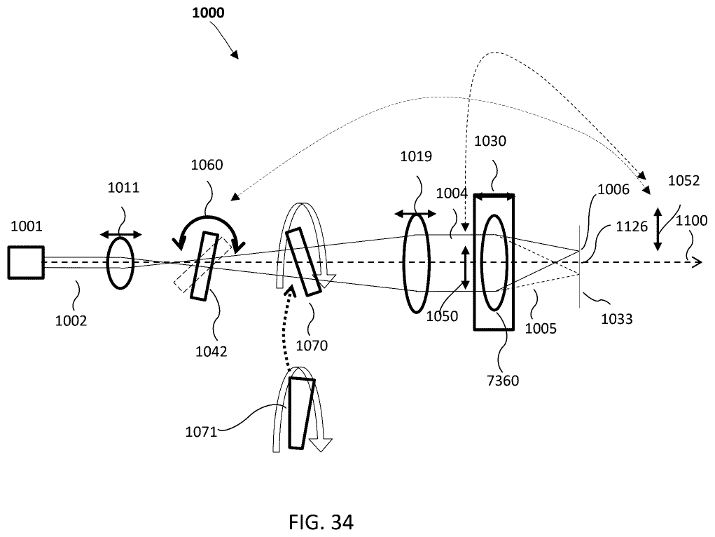

FIG. 34 shows another preferred embodiment of the laser scanning system. Here the circular scan is no longer using a rotating focusing lens but is now performed by a rotating tilted plate 1070 that spins/rotates around the optical axis 1100. Each complete 360 deg rotation results in a focus circle scan in the focal plane 1033. The radius of the scan circle depends on the thickness of the tilted plate and its tilt angle. The exact optics settings are calculated using a optical ray tracing software program. Typical tilt angles of around 1-3 degrees from normal incidence and a plate thickness of 6 mm (BK7) will result in a focus scan circle diameter of 40 μm to 100 μm for a focusing numerical aperture of around 0.1 and thereby create a capsule scanning pattern as shown in FIG. 33d right side. For other applications and target areas, larger or smaller circles scans can be achieved by tilting the rotating/spinning plate 1070 between 0 and 80 degrees. To maintain low aberrations the tilt angle should be limited to under 30 degrees. The translation part of the scanning system (resulting in the capsule pattern) is here still performed by translating 1050 the focusing lens unit 1030 or adjusting (rotating) the tilt angle 1060 of the tilted plate 1042, same as in FIG. 33. A rotating wedge (prism) 1071 can also be used instead of the rotating tilted plate 1070. The wedge angle determines the amount of focus shift and therefore the circular scan diameter. For the configuration described in this chapter a wedge angle of 0.5 degrees will result in a scan circle diameter between 50 μm and 150 μm. Larger wedge angles up to 20 degrees can be used for bigger circular scan diameters. To maintain low aberrations, wedge angles up to 5 degrees are preferred.

Alternatively, a gimbal mirror system as described starting in line 357 can also be used to perform the here disclosed circular scanning patterns and or lateral translation. Another delivery system approach to create these scanning patterns uses galvo, servo or MEMS based mirror scanners.

### Significant Advantages of the Presently Disclosed Systems and Methods

The present disclosure provides novel systems and methods for treating glaucoma and other eye disorders. The presently disclosed systems and methods provide substantial benefits and advantages over conventional systems and methods.

For example, in accordance with some embodiments, the presently disclosed systems and methods provide versatile scanning techniques that can create numerous patterns and shapes, with specific laser spot sizes and power. The presently disclosed systems and methods provide novel rotating optics, such as wedges, tilted parallel plates, or offset lenses, that enable circular, ring, spiral, or partial coverage patterns for full or partial hole creation.

In accordance with some embodiments, the presently disclosed systems and methods are far less complex than conventional systems and methods. This enables the presently disclosed systems and methods to be performed while being insensitive to small misalignments, often avoiding the bulky galvo mirror systems or large correction optics.

In accordance with some embodiments, the presently disclosed systems and methods also enable the creation of a novel “soft hole” in treating the eye. In forming a “soft hole,” the system can beneficially implement only partially disruptive scanning and spare substantial tissue volume while achieving fluid flow enhancement and tissue remodeling. By preserving tissue of the trabecular meshwork, the native pathways can be reopened and reinvigorated, thereby preserving as much normal function of the trabecular meshwork as possible. This is a substantial benefit compared to conventional methods that obliterate or entirely native trabecular meshwork tissue with bulky tubular devices.

In accordance with some embodiments, the presently disclosed systems and methods also enable the use of integrated imaging. The systems and components disclosed herein can incorporate onboard goniolens/camera modules, OCT, and/or two-photon detection to allow real-time localization and feedback on tissue thickness and location.

In accordance with some embodiments, the presently disclosed systems and methods provide scalable energy and spot size customization. The systems and methods support a range of laser energies (tens to hundreds of μJ) and spot sizes from a few microns to tens of microns, which allows a user to customize the treatment based on the target area and patient considerations, providing incredible versatility compared to conventional solutions.

In accordance with some embodiments, the presently disclosed systems and methods can also provide z-axis scanning or optional lens translation or offset rotation in order to provide volumetric ablation for creating channels or partial thickness cuts.

In accordance with some embodiments, the presently disclosed systems and methods can also be repeated on a patient far more than conventional treatment options. This outstanding possibility is primarily due to the customizability of the presently disclosed treatments, such as by providing partial coverage approach and due to mild photodisruption that advantageously results in reduce scarring.

In accordance with some embodiments, the presently disclosed systems and methods also provide a user-friendly integration for the user. For example, combined camera-goniolens units simplify in-procedure visualization and reduce operator overhead.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An ophthalmic laser treatment apparatus, comprising: a laser source configured to emit pulses between about 100 fs and 50 ns at energies up to about 500 μJ; a beam-expanding telescope to produce a collimated beam of at least 5 mm diameter; and a first rotating optical scanner selected from a wedge, tilted parallel plate, or offset lens, adapted to create a circular or ring scanning trajectory.

Clause 2. The apparatus of Clause 1, further comprising a second rotating optical scanner in series, each scanner operating at a distinct rotation speed.

Clause 3. The apparatus of any of Clauses 1-2, wherein the laser source operates at about 532 nm.

Clause 4. The apparatus of any of Clauses 1-3, wherein the collimated beam diameter is at least 10 mm, enabling sub-10 μm spot focusing.

Clause 5. The apparatus of any of Clauses 1-4, further comprising a motorized stage to translate a focusing lens in the Z-axis for volumetric scanning.

Clause 6. The apparatus of any of Clauses 1-5, wherein the goniolens includes a camera integrated behind a partially reflective mirror for real-time angle visualization.

Clause 6.5. The apparatus of any of Clauses 1-6, further comprising a patient interface with a goniolens for delivering the scanning beam into the eye's anterior angle, wherein the apparatus is adapted to form openings or partial openings in the trabecular meshwork with reduced aberrations.

Clause 7. A laser delivery system for generating partial-coverage soft holes in the trabecular meshwork, comprising: a pulsed laser source operating at energies of about 30-300 μJ; a scanning assembly configured to trace a low-density pattern of spots over a diameter of 100-600 μm; and a focusing lens providing a spot size under 10 μm.

Clause 8. The apparatus of Clause 7, wherein the scanning assembly further comprises rotating wedges at different rotation frequencies, yielding a sparse circular pattern with <50% coverage.

Clause 9. The apparatus of any of Clauses 7-8, wherein the focusing lens is integrated with a lens offset motor for lateral scanning.

Clause 10. The apparatus of any of Clauses 7-9, further comprising an OCT subsystem that registers the exact depth of the meshwork.

Clause 11. The apparatus of any of Clauses 7-10, wherein the partial-coverage pattern is formed by enabling laser pulses only along arc segments of each circle.

Clause 12. The apparatus of any of Clauses 7-11, further comprising a user interface that sets partial coverage by adjusting scanning speed relative to the pulse repetition rate.

Clause 12.5. The apparatus of any of Clauses 7-12, further comprising a patient interface lens contacting the cornea, wherein the partial-coverage pattern disrupts only a fraction of the trabecular meshwork tissue, leaving most tissue volume intact while enhancing outflow.

Clause 13. A laser system for ophthalmic surgery, comprising: a beam shaping unit including at least one tilted parallel plate that rotates to deviate a pulsed laser beam; a second scanning unit configured to shift the beam in a perpendicular axis or a Z-axis direction; a gonioscopic lens having an integrated camera for angle visualization; and control electronics that synchronize rotation speeds and lens translation, wherein the system performs ring scanning with variable radii by adjusting the plate tilt and lens position in real time.

Clause 14. The apparatus of Clause 13, wherein the second scanning unit is a galvanometric mirror that fine-tunes the spot's lateral position.

Clause 15. The apparatus of any of Clauses 13-14, further comprising a two-photon detection channel for measuring tissue fluorescence or second-harmonic generation.

Clause 16. The apparatus of any of Clauses 13-15, wherein the integrated camera is a high-sensitivity digital sensor capturing the trabecular meshwork in low-light conditions.

Clause 17. The apparatus of any of Clauses 13-16, wherein the control electronics maintain a constant shot spacing by dynamically adjusting rotation speeds.

Clause 18. The apparatus of any of Clauses 13-17, further comprising a user interface that displays real-time camera imagery alongside scanning parameters.

Clause 19. A rotational lens-offset scanning apparatus for creating ring or spiral ablations in an eye, comprising: a focusing lens offset from the central optical axis; a rotation stage spinning the lens about the axis, thereby tracing a circular path at the focal plane; and a beam expansion system delivering a collimated beam to the offset lens; wherein the apparatus is configured to produce ring diameters from about 50 μm to about 1 mm.

Clause 20. The apparatus of Clause 19, further comprising a second rotation stage or wedge to superimpose an additional offset, forming complex scanning shapes.

Clause 21. The apparatus of any of Clauses 19-20, wherein the lens offset is adjustable by a motor to vary ring radius.

Clause 22. The apparatus of any of Clauses 19-21, wherein the focusing lens is an aspheric lens to minimize off-axis aberrations.

Clause 23. The apparatus of any of Clauses 19-22, further comprising a diagnostic camera that confirms ring placement in relation to the scleral spur.

Clause 24. The apparatus of any of Clauses 19-23, wherein the scanning motion is synchronized with a shutter to create segmented arcs.

Clause 24.5. The apparatus of any of Clauses 19-24, further comprising a patient interface that seats against the cornea and guides the scanning beam toward the trabecular meshwork.

Clause 25. A combined camera-goniolens unit for laser-based eye treatments, comprising: a goniolens with a reflective or partially reflective surface angled to view the anterior angle; a digital camera mounted behind the surface to capture images of the trabecular meshwork; an optical port for introducing a pulsed laser beam through or around the goniolens; and an interface allowing the camera-goniolens unit to dock with or detach from a main laser system, wherein the combined unit facilitates real-time angle visualization and alignment of the treatment beam with minimal device complexity.

Clause 26. The apparatus of Clause 25, further comprising an illumination source for highlighting the pigmented meshwork.

Clause 27. The apparatus of any of Clauses 25-26, wherein the camera is oriented at a small offset angle to the laser axis for direct image capture.

Clause 28. The apparatus of any of Clauses 25-27, further comprising a suction ring to stabilize the goniolens on the cornea.

Clause 29. The apparatus of any of Clauses 25-28, wherein the partial reflective surface transmits near-infrared light to the camera while reflecting the 532 nm treatment beam.

Clause 30. The apparatus of any of Clauses 25-29, further comprising an integrated battery and wireless transmitter for portable or handheld usage.

Clause 31. A laser scanning system for volumetric partial ablation in the trabecular meshwork, comprising: a pulsed laser source delivering pulses between about 10 Hz and 5 kHz; a multi-axis scanning unit capable of shifting the focal spot in X, Y, and Z directions; a goniolens or contact lens arrangement enabling direct anterior angle access; and a controller that defines multiple depth layers for scanning, wherein the system forms layered ablations from near Schlemm's canal outward, or vice versa, to create a channel through the meshwork.

Clause 32. The apparatus of Clause 31, wherein the scanning unit includes a linear motor for incremental Z-step movement.

Clause 33. The apparatus of any of Clauses 31-32, further comprising an OCT module to measure real-time tissue thickness at each depth layer.

Clause 34. The apparatus of any of Clauses 31-33, wherein the pulses are at 532 nm and energy about 50-150 PJ.

Clause 35. The apparatus of any of Clauses 31-34, wherein the partial ablation includes a soft hole approach, sparing at least half the tissue volume.

Clause 36. The apparatus of any of Clauses 31-35, wherein the system logs scanning patterns and depth data for follow-up procedures.

Clause 37. A glaucoma-treatment apparatus comprising: a laser engine generating pulses of about 1-20 ns at wavelengths from 400-650 nm; a scanning subassembly with at least two rotating optical elements producing a compound offset for ring scanning patterns; a goniolens with an optional cutout region to permit external imaging beams to access the limbus region; and a software-controlled user interface that selects ring diameter, scanning density, or partial coverage, wherein the apparatus allows creation of hole(s) in the trabecular meshwork with modifiable scanning coverage for customizing outflow enhancement.

Clause 38. The apparatus of Clause 37, wherein the second rotating element is a lens offset mount that superimposes an eccentric trajectory on the beam.

Clause 39. The apparatus of any of Clauses 37-38, further comprising a lighting unit to constrict the iris via retinal illumination.

Clause 40. The apparatus of any of Clauses 37-39, wherein partial coverage is automatically calculated based on user-input coverage ratio.

Clause 41. The apparatus of any of Clauses 37-40, wherein the scanning subassembly is enclosed in a sterilizable housing.

Clause 42. The apparatus of any of Clauses 37-41, wherein the system stores scanning logs for each patient procedure.

Clause 43. A handheld camera-goniolens scanning device, comprising: a goniolens body shaped to contact the cornea and expose the anterior angle; a digital camera module embedded within or adjacent to the goniolens body; a low-power laser scanning path that enters the goniolens off-axis; and an integrated motor that rotates a focusing or offset lens to trace a partial ring or arc pattern, wherein the device is operable by hand to both visualize and treat trabecular meshwork tissue.

Clause 44. The apparatus of Clause 43, further comprising a handle portion at least 20 mm long, enabling easy handheld manipulation.

Clause 45. The apparatus of any of Clauses 43-44, wherein the scanning path is deflected by a mirror integrated behind the camera sensor.

Clause 46. The apparatus of any of Clauses 43-45, further comprising a micro display or HUD showing real-time camera output.

Clause 47. The apparatus of any of Clauses 43-46, wherein the integrated motor spins at 1-50 Hz for partial ring coverage.

Clause 48. The apparatus of any of Clauses 43-47, further comprising a battery-powered laser source with energies up to 50 μJ per pulse.

Clause 49. A laser apparatus with two-photon detection for mapping the trabecular meshwork, comprising: a pulsed laser source delivering sub-10 ps pulses at energies about 20-100 μJ; a scanning lens offset assembly generating circular or spiral scans; and a two-photon fluorescence or second-harmonic detection channel aligned to capture backscattered signals; wherein the detection channel provides real-time imaging of meshwork structures and depth prior to or during ablation.

Clause 50. The apparatus of Clause 49, further comprising a filter passing only wavelengths corresponding to the two-photon emission.

Clause 51. The apparatus of any of Clauses 49-50, wherein the scanning lens offset is adjustable to shift ring diameter from 50 μm to 500 μm.

Clause 52. The apparatus of any of Clauses 49-51, wherein the detection channel is used at reduced laser power to map tissue before switching to higher power for ablation.

Clause 53. The apparatus of any of Clauses 49-52, wherein the system logs intensity signals correlated with each XY coordinate.

Clause 54. The apparatus of any of Clauses 49-53, further comprising a user interface that displays 2D or 3D reconstructions of the scanned angle region.

Clause 54.5. The apparatus of any of Clauses 49-54, further comprising a goniolens-based patient interface that seats on the cornea.

Clause 55. An ophthalmic laser system with integrated camera-lens subassembly, comprising: a primary laser source for photodisruption at pulse energies of 40-300 μJ; a beam-shaping unit with at least one rotating wedge to create spiral or ring coverage of the anterior angle; a camera-lens assembly in line with or slightly offset from the laser beam path, capturing real-time images through a partially reflective goniolens mirror; and a footswitch or hand switch controlling both laser firing and scanning speed, wherein the integrated camera-lens subassembly reduces the need for external slit lamp or standalone microscope.

Clause 56. The apparatus of Clause 55, wherein the footswitch includes tactile or audio feedback.

Clause 57. The apparatus of any of Clauses 55-56, further comprising an alignment laser distinct from the treatment laser for calibrating the meshwork position.

Clause 58. The apparatus of any of Clauses 55-57, wherein the ring coverage is automatically computed based on user-specified diameter and depth.

Clause 59. The apparatus of any of Clauses 55-58, wherein the partially reflective mirror transmits at least 80% of visible wavelengths to the camera.

Clause 60. The apparatus of any of Clauses 55-59, further comprising a data port that transfers recorded images and scanning logs to an external device.

Clause 61. A laser apparatus configured for selective partial-thickness ablation of the trabecular meshwork, comprising: a scanning system that can deliver pulses in at least two Z-axis layers; a beam control mechanism to restrict ablation depth to a fraction of the total meshwork thickness; a goniolens unit allowing direct or angled visualization of the meshwork; and an operator console that sets each Z-layer's scanning radius and coverage, wherein the system partially ablates an inner or outer portion of the mesh, leaving the remainder intact.

Clause 62. The apparatus of Clause 61, wherein the system is programmed to commence scanning from Schlemm's canal outward.

Clause 63. The apparatus of any of Clauses 61-62, wherein a sensor detects backscattered signals to confirm layer thickness.

Clause 64. The apparatus of any of Clauses 61-63, further comprising a shutter that toggles laser pulses at each Z-layer boundary.

Clause 65. The apparatus of any of Clauses 61-64, wherein the console displays depth increments of 5-50 μm.

Clause 66. The apparatus of any of Clauses 61-65, wherein the partial thickness approach is repeated in multiple arcs for incremental meshwork removal.

Clause 67. A compact scanning-laser apparatus for minimal corneal footprint, comprising: a diode-pumped laser generating subthreshold or photodisruptive pulses at 50-500 μJ; a rotating mirror or wedge-based scanner with an optical axis nearly parallel to the cornea; a small-form-factor goniolens camera assembly attached near the corneal apex; and a mechanical structure that allows rotation around the eye's central axis for 360° coverage of the angle, wherein the apparatus occupies minimal space above the patient's eye while enabling full-angle scanning.

Clause 68. The apparatus of Clause 67, wherein the rotating wedge extends no more than 25 mm above the corneal surface.

Clause 69. The apparatus of any of Clauses 67-68, further comprising a vacuum ring that holds the goniolens firmly.

Clause 70. The apparatus of any of Clauses 67-69, wherein the user can rotate the entire assembly by hand around a vertical axis.

Clause 71. The apparatus of any of Clauses 67-70, wherein a flexible cable supplies power and control signals to the scanning-laser apparatus.

Clause 72. The apparatus of any of Clauses 67-71, further comprising a small integrated display showing the meshwork area on a 2-3 inch screen.

Clause 73. A method of ring-based laser scanning for glaucoma treatment, comprising: expanding a pulsed laser beam to at least about 10 mm diameter, rotating a wedge or offset lens about a central axis to form a ring pattern in the trabecular meshwork, focusing the beam onto the meshwork via a goniolens interface, and optionally adjusting ring diameter or partial coverage by altering rotation speed or wedge tilt, whereby a ring of ablation is created to enhance aqueous humor outflow.

Clause 74. The method of Clause 73, wherein partial coverage is achieved by shuttering the laser for only a segment of each ring revolution.

Clause 75. The method of any of Clauses 73-74, further comprising defocusing the ring plane to expand spot size from ˜5 μm to ˜30 μm.

Clause 76. The method of any of Clauses 73-75, wherein the ring diameter is incrementally enlarged after each pass to gradually expand the ablated zone.

Clause 77. The method of any of Clauses 73-76, including verifying ring placement via a camera integrated in the goniolens.

Clause 78. The method of any of Clauses 73-77, further comprising applying multiple ring layers at different depths in the mesh.

Clause 79. A method of creating a soft hole in the trabecular meshwork, comprising: providing a pulsed laser at about 40-200 μJ per pulse, generating a low-density scanning pattern over a ˜400 μm diameter region of the mesh, restricting coverage so that <50% of the tissue is disrupted, and observing the partial ablation via a goniolens or integrated camera, wherein the resultant soft hole disrupts only a fraction of the meshwork thickness, preserving structural integrity.

Clause 80. The method of Clause 79, further comprising applying 2-5 such soft holes around the angle circumference.

Clause 81. The method of any of Clauses 79-80, wherein the scanning pattern is arcs, each occupying <60° of a full circle.

Clause 82. The method of any of Clauses 79-81, wherein the user sets coverage density through a graphical interface.

Clause 83. The method of any of Clauses 79-82, further including verifying photodisruption in real time with a two-photon detection channel.

Clause 84. The method of any of Clauses 79-83, wherein partial coverage is repeated at multiple angles to produce multiple soft holes.

Clause 85. A method of Z-axis layer scanning for trabecular meshwork ablation, comprising: measuring the meshwork thickness or location using OCT or confocal imaging, setting multiple depth planes from near Schlemm's canal outwards, scanning the laser focal spot at each plane, removing or partially removing the meshwork layer, and repeating step (3) for subsequent planes, whereby a channel is formed that may be partial or full thickness.

Clause 86. The method of Clause 85, further comprising applying a shutter to skip certain planes, yielding a stepped ablation profile.

Clause 87. The method of any of Clauses 85-86, wherein the spot spacing at each plane is set to 1-20 μm.

Clause 88. The method of any of Clauses 85-87, wherein real-time feedback from the imaging system automatically halts scanning upon reaching Schlemm's canal.

Clause 89. The method of any of Clauses 85-88, further comprising rotating the goniolens or mirror about the eye's axis for coverage in multiple quadrants.

Clause 90. The method of any of Clauses 85-89, wherein partially ablating each plane yields a soft hole at each depth.

Clause 91. A method of merging ring scanning with partial arcs to tailor outflow, comprising: rotating a lens offset or wedge to create a ring focus in the meshwork, pulsing the laser only on partial arcs of the ring so as to form multiple radial slits in the mesh, shifting the ring or arcs laterally across a target zone, and optionally defocusing or adjusting energy to expand/or reduce spot diameter, wherein the user tailors outflow by controlling the number and width of radial arcs.

Clause 92. The method of Clause 91, further comprising selecting an arc coverage fraction from 10% to 90%.

Clause 93. The method of any of Clauses 91-92, wherein the ring is ˜200 μm in diameter, shifted incrementally across a 1 mm zone.

Clause 94. The method of any of Clauses 91-93, further comprising camera-based verification of each radial slit before moving on.

Clause 95. The method of any of Clauses 91-94, wherein the ring speed is set at 20 Hz, and the laser repetition rate at 2 kHz.

Clause 96. The method of any of Clauses 91-95, including a final pass that connects some arcs to Schlemm's canal, forming a continuous opening.

Clause 97. A method of performing partial-thickness ablation in the eye's trabecular meshwork from inside outward, comprising: positioning a goniolens on the cornea, focusing a pulsed laser at an initial depth near Schlemm's canal, scanning a series of arcs or rings that ablate only up to a specified fraction of the total meshwork thickness, optionally rotating the scanning unit to shift the ablation zone across the angle, whereby only the deeper portion of the meshwork is removed, leaving the outer layers intact.

Clause 98. The method of Clause 97, further comprising an OCT or confocal imaging step verifying the ablation depth.

Clause 99. The method of any of Clauses 97-98, wherein the arcs are formed by a rotating wedge spinning at 10-50 Hz.

Clause 100. The method of any of Clauses 97-99, further comprising pulsing at 1-10 kHz repetition rate for rapid scanning.

Clause 101. The method of any of Clauses 97-100, wherein the goniolens incorporates a camera that displays progress in real time.

Clause 102. The method of any of Clauses 97-101, wherein multiple passes are performed at increasing energies to enlarge the partial ablation zone.

Clause 103. A method of handheld scanning in the anterior angle, comprising: securing a handheld goniolens with an embedded mirror and camera against the cornea, directing a pulsed laser beam at an offset input port on the device, rotating a small offset lens or mirror within the handheld device to create a localized circular or elliptical ablation path, capturing real-time images on the camera for verifying alignment, whereby the operator can manually position and angle the device to ablate selective zones of the mesh.

Clause 104. The method of Clause 103, wherein the handheld device includes a mechanical dial that changes the offset lens radius.

Clause 105. The method of any of Clauses 103-104, further comprising using a footswitch to fire individual pulses as the operator sees fit.

Clause 106. The method of any of Clauses 103-105, wherein the elliptical ablation path is created by tilting the lens axis relative to the laser beam.

Clause 107. The method of any of Clauses 103-106, further comprising applying partial coverage by turning the beam on/off for segments of each elliptical pass.

Clause 108. The method of any of Clauses 103-107, wherein an onboard battery powers the scanning motor and camera.

Clause 109. A method of two-photon guided ablation of the trabecular meshwork, comprising: operating a pulsed laser in a lower-energy scanning mode to generate two-photon fluorescence or second harmonic signals; mapping the mesh's structural features in real time; switching the laser to a higher-energy mode for actual ablation along the mapped trajectory; and verifying ablation extent by periodically reverting to the lower-energy scanning mode, wherein real-time two-photon detection guides the ablation to precise tissue layers.

Clause 110. The method of Clause 109, further comprising capturing a 3D map of Schlemm's canal boundary to avoid ablating that structure.

Clause 111. The method of any of Clauses 109-110, wherein the ablation pattern is circular and the scanning lens offset is adjusted after mapping.

Clause 112. The method of any of Clauses 109-111, wherein the user interface toggles between map mode and ablation mode automatically.

Clause 113. The method of any of Clauses 109-112, including storing the mapped data for post-procedure analysis.

Clause 114. The method of any of Clauses 109-113, wherein partial coverage is used if real-time mapping indicates thin meshwork regions.

Clause 115. A method of scanning-based “full hole” creation in the meshwork, comprising: placing a goniolens on the cornea, setting a scanning pattern that overlaps pulses densely across a specified zone, delivering sufficient pulse energy (>100 μJ) to perforate the meshwork from the inner to outer boundary, optionally rotating the scanning pattern for multiple adjacent zones, whereby a complete hole is formed through the trabecular meshwork into Schlemm's canal.

Clause 116. The method of Clause 115, further comprising adjusting the scanning overlap to ensure near-continuous ablation across the zone.

Clause 117. The method of any of Clauses 115-116, wherein a camera-lens assembly verifies “breakthrough” at the canal boundary.

Clause 118. The method of any of Clauses 115-117, wherein each zone is about 300 μm in diameter, repeated around the angle if needed.

Clause 119. The method of any of Clauses 115-118, further comprising applying final low-power pulses to smooth the channel edges.

Clause 120. The method of any of Clauses 115-119, wherein an integrated imaging unit monitors bubble formation or tissue egress to confirm a full perforation.

Clause 121. An ophthalmic laser treatment apparatus, comprising: a laser source configured to emit a laser beam along an original beam axis; a beam-expanding telescope having a rotating optic that is adapted to create a circular or capsule shaped scanning trajectory that is offset from the original beam axis when a laser beam passes through the beam-expanding telescope.

Clause 122. The apparatus of Clause 121, wherein the beam-expanding telescope is configured to produce a collimated beam of at least 5 mm diameter.

Clause 123. The apparatus of any of Clauses 121-122, wherein the rotating optic comprises a tilted parallel plate or an offset lens.

Clause 124. The apparatus of any of Clauses 121-123, wherein the beam-expanding telescope further comprises a second rotating optic in series with the rotating optic.

Clause 125. The apparatus of any of Clauses 121-124, wherein the rotating optic and the second rotating optic rotate at different rotation speeds.

Clause 126. The apparatus of any of Clauses 121-125, wherein the rotating optic and the second rotating optic each rotate about respective rotational axes that are offset from or noncolinear with the original beam axis.

Clause 127. The apparatus of any of Clauses 121-126, wherein the beam-expanding telescope comprises a translatable optic for adjusting a z-axis depth of a focus point of the laser beam on a focal plane thereof.

Clause 128. The apparatus of any of Clauses 121-127, wherein the beam-expanding telescope comprises a tiltable optic for translating the focus point of the laser beam on the focal plane thereof.

Clause 129. The apparatus of any of Clauses 121-128, wherein the tiltable optic is configured to linearly translate the focus point of the laser beam on the focal plane thereof.

Clause 130. The apparatus of any of Clauses 121-129, wherein the laser source operates at about 532 nm.

Clause 131. The apparatus of any of Clauses 121-130, further comprising a patient interface with a goniolens for delivering the scanning beam into the eye's anterior angle, wherein the apparatus is adapted to form openings or partial openings in the trabecular meshwork with reduced aberrations.

Clause 132. The apparatus of Clause 131, wherein the goniolens includes a camera integrated behind a partially reflective mirror for real-time angle visualization.

Clause 133. An apparatus that incorporates any of the features of the preceding Clauses.

Clause 134. A method that performs aspects of any of the methods or apparatuses of the preceding Clauses.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

1. An ophthalmic laser treatment apparatus, comprising:
a laser source configured to emit pulses between about 100 fs and 50 ns at energies up to about 500 μJ;
a beam-expanding telescope to produce a collimated beam of at least 5 mm diameter; and
a first rotating optical scanner selected from a tilted parallel plate, or optical wedge, or offset lens, adapted to create a circular or ring scanning trajectory;
wherein the apparatus is adapted to form openings or partial openings in the trabecular meshwork with reduced aberrations.

2. The apparatus of claim 1, further comprising a second rotating optical scanner in series, each scanner operating at a distinct rotation speed.

3. The apparatus of claim 1, further comprising a motorized translation stage to translate a focusing lens in the x-y axis plane to expand the circular scan to a capsule shaped scanning pattern area.

4. The apparatus of claim 1, wherein the collimated beam diameter is at least 10 mm, enabling sub-15 μm spot focusing.

5. The apparatus of claim 1, further comprising a motorized stage to translate a focusing or telescope lens in the Z-axis for volumetric scanning.

6. The apparatus of claim 1, further comprising a motorized rotation stage to tilt a parallel optical plate around a x or y axis to expand the circular scan to a capsule shaped scanning pattern area.

7. The apparatus of claim 1, further comprising a patient interface with a goniolens for delivering the scanning beam into the eye's anterior angle.

8. A laser delivery system for generating partial-coverage soft holes in the trabecular meshwork, comprising:
a pulsed laser source operating at energies of about 10-300 μJ;
a scanning assembly configured to trace a low-density pattern of spots over a diameter of 50-600 μm;
a focusing lens providing a spot size under 20 μm; and
wherein the partial-coverage pattern disrupts only a fraction of the trabecular meshwork tissue, leaving most tissue volume intact while enhancing outflow.

9. The apparatus of claim 8, wherein the scanning assembly further comprises rotating wedges at different rotation frequencies, yielding a sparse circular pattern with <50% coverage.

10. The apparatus of claim 8, wherein the focusing lens is integrated with a lens offset motor for lateral scanning.

11. The apparatus of claim 8, further comprising an OCT subsystem that registers the exact depth of the meshwork.

12. The apparatus of claim 8, wherein the partial-coverage pattern is formed by enabling laser pulses only along arc segments of each circle.

13. The apparatus of claim 8, further comprising a user interface that sets partial coverage by adjusting scanning speed relative to the pulse repetition rate.

14. The apparatus of claim 8, further comprising a patient interface lens for contacting the cornea.

15. A laser system for ophthalmic surgery, comprising:
a beam shaping unit including at least one tilted parallel plate that rotates to deviate a pulsed laser beam;
a second scanning unit configured to shift the beam in a perpendicular axis or a Z-axis direction; and
control electronics that synchronize rotation speeds and lens translation, wherein the system performs ring scanning with variable radii by adjusting the plate tilt and lens position in real time.

16. The apparatus of claim 15, wherein the second scanning unit is a galvanometric mirror that fine-tunes the spot's lateral position.

17. The apparatus of claim 15, further comprising a two-photon detection channel for measuring tissue fluorescence or second-harmonic generation.

18. The apparatus of claim 15, wherein the integrated camera is a high-sensitivity digital sensor capturing the trabecular meshwork in low-light conditions.

19. The apparatus of claim 15, wherein the control electronics maintain a constant shot spacing by dynamically adjusting rotation speeds.

20. The apparatus of claim 15, further comprising a gonioscopic lens having an integrated camera for angle visualization and/or a user interface that displays real-time camera imagery alongside scanning parameters.
